(19) United States
(12) Patent Application Publication
(10) Pub. No.: US 2005/0245738 A1
(43) Pub. Date: Nov. 3, 2005

(54) Stable bioavailable crystalline form or cefdinir and a process for the preparation thereof

(75) Inventors: Girij Pal Singh, Pune (IN); Himadri Sen, Pune (IN); Dhananjai Srivastava, Pune (IN); Himanshu Madhav Godbole, Pune (IN); Gurvinder Pal Singh, Pune (IN); Pravin Raghunath Mahajan, Pune (IN); Umesh Babanrao Rananaware, Pune (IN); Sagar Purushottam Nehate, Pune (IN); Sanjay Chhagan Wagh, Pune (IN)

Correspondence Address: Merchant & Gould P.C., P.O. Box 2903, Minneapolis, MN, 55402-0903, US

(73) Assignee: LUPIN LTD, Mumbai (IN)

(21) Appl. No.: 10/838,431

(22) Filed: May 3, 2004

(51) Int. Cl. A61K 31/545; C07D 501/18

(52) U.S. Cl. 540/222

(57) ABSTRACT

The present invention relates to a stable and bioavailable crystalline form of a third generation cephalosporin antibiotic, cefdinir and a process for the preparation thereof. The present invention also relates to a pharmaceutical composition containing the novel crystalline cefdinir, useful in the treatment of maladies such as bacterial infections.

The present invention relates to a stable and bioavailable crystalline form of a third generation cephalosporin antibiotic, cefdinir and a process for the preparation thereof. The present invention also relates to a pharmaceutical composition containing the novel crystalline cefdinir, useful in the treatment of bacterial infections.

### BACKGROUND OF THE INVENTION

The chemical entity, (Z)-7-[2-(2-amino-4-thiazolyl)-2-hydroxyiminoacetamido]-3-vinyl-3-cephem-4-carboxylic acid of formula (I), generically known as cefdinir is a therapeutically and commercially valuable third generation cephalosporin antibiotic for oral administration.

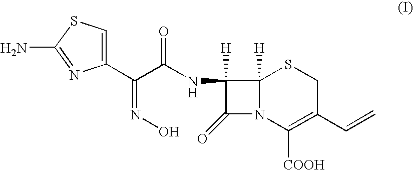

Cefdinir is effective against both gram-positive and gram-negative bacteria and has been found to have good stability towards β-lactamases. It is particularly effective against S. aureus, which has shown resistance to other oral cephalosporins. Cefdinir is used in the treatment of chronic bronchitis, acute maxillary sinusitis, pharyngitis/tonsillitis, community acquired pneumonia caused by H. influenzae and uncomplicated skin and skin-structure infections caused by S. aureus and S. pyogenes.

Cefdinir was approved by the US FDA on Dec. 4, 1997 and is marketed as capsules and suspension for oral administration under the brand name Omnicef®.

Cefdinir was first disclosed by Takaya et. al. in U.S. Pat. No. 4,559,334 (hereinafter “the '334 patent”), which also describes a method for synthesis of the drug substance. Examples-14 and 16 of the '334 patent describe the synthesis and isolation of cefdinir having the following IR spectral values:
IR (Nujol) cm⁻¹: 3300, 1780, 1665, 1180, 1130
   However, this IR spectrum is the only physical information disclosed in the '334 patent which can be used to identify compounds produced under the methods disclosed therein. There is no mention whatsoever in the specification about the crystal nature of the product obtained therein. In other words, the '334 patent does not contain any details of the solid state characteristics of cefdinir prepared, such as X-ray (powder) diffraction pattern, DSC thermogram, TGA thermogram, or solid state NMR spectrum, apart from mere mention of its IR spectra. In other words, the '334 patent does not state whether the cefdinir produced is crystalline, semi-crystalline, or amorphous.

Another crystalline form of cefdinir and a process for preparation thereof is disclosed by Takaya et al. in U.S. Pat. No. 4,935,507 (hereinafter “the '507 patent”). The '507 patent claims that cefdinir prepared as per the method disclosed in Examples 14 and 16 of the '334 patent is a “crystalline like amorphous product, not a crystalline product,” which has several disadvantages in that “it is bulky, not as pure, unstable and insufficient in filtration rate.” These properties render the material not particularly amenable for pharmaceutical preparations, difficult to use in a large-scale production, and cause difficulty in storage.

The '507 patent states that the process disclosed therein provides a crystalline form of cefdinir, designated as “Crystal A”, which is distinctly different from the material obtained by the method disclosed in the '334 patent in that:
a. It exhibits a characteristic IR Spectrum spectrum having the following peaks,

IR (Nujol) cm⁻¹: 1760, 1670, 1620

which are distinctly different from the values given for cefdinir in Examples 14 and 16 of the '334 patent;

b. It possesses a characteristic X-ray (powder) diffraction; which presumably differs from the “crystalline like amorphous product” disclosed in the '334 patent (albeit not reported in the patent);
c. It has a lower bulk density in comparison to the product obtained by the method of the '334 patent; and
d. It is pure and stable against heat and light and therefore, more amenable for formulation into a suitable dosage form.

The characteristic peaks of the IR spectrum for the Crystal A form of cefdinir are distinctly different from the values given for cefdinir in Examples 14 and 16 of the '334 patent. Nonetheless, the '334 patent and the '507 patent use very similar techniques for the synthesis of cefdinir, with the '507 patent further crystallizing the compound prepared under Examples 14 and 16 of the '334 patent, or its alkali metal salt, to obtain a new crystalline form of cefdinir, Crystal A.

Other methods for synthesis of cefdinir have been discovered. U.S. Pat. No. 6,093,814 (Lee et. al.); EP 1 340 751 A1 (Ono et. al.); U.S. Pat. No. 6,350,869 B1 (Sturm et. al.); EP 1 273 587 A1 (Kamayema et. al.); WO 02/098884 A1 (Lee et. al.); and WO 03/091261 A1 (Kumar et. al.). Most of these references, like the '334 patent, characterize the product by its respective IR spectrum, all of which, incidentally, are rather similar. This suggests that the products produced under the methods of these patents possess the same solid-state properties.

Recently, however, U.S. Application No. 2003/0204082 A1 laid claims to a purportedly novel form of crystalline cefdinir, stating the following:
a. It provides a new crystalline form of cefdinir having a characteristic X-ray (powder) diffraction pattern, which is prepared by adding an organic solvent selected from either ethyl acetate or tetrahydrofuran to an aqueous solution containing cefdinir produced according to the method disclosed in the '334 patent, cooling the solution to a temperature between 0° C. to 6° C., and lowering the pH to between 1.5 and 3.0 to cause precipitation of the new crystalline form of cefdinir, which is isolated by known techniques, e.g. filtration;
b. The new crystalline form thus produced has a water content of 6.0%, which suggests that it has 1.5 molecules of water in the crystal lattice, i.e. it is a sesquihydrate of cefdinir. The patent application also mentions that the crystalline material has a titre value of 949 ig/mg on anhydrous basis and the total impurities present therein are 0.10%;
c. On accelerated stability tests the stability of the new crystalline form was found to be at least equal to that of the reference “Crystal A” disclosed in the '507 patent. The dissolution rate of the new crystalline form in 0.07 N Hydrochloric acid was found to be initially slower than dissolution rate exhibited by the reference “Crystal A”, however, the patent application mentions that upon prolonging the duration of the dissolution treatment it was found that after the first hour, the profile changed, wherein the new crystalline form was found to be more soluble than the reference “Crystal A.”

Most of the methods heretofore disclosed require the use of solvents like ethyl acetate, tetrahydrofuran, dichloromethane, and the like, during crystallization and isolation of cefdinir. Ethyl acetate and tetrahydrofuran have a low flash point of 26° F. (−3° C.) and 1° F. (−17° C.) respectively. Consequently, these solvents pose serious hazards in operability of the process. In addition, tetrahydrofuran and dichloromethane have been classified as Class II solvents by the International Conference on Harmonization (ICH), and are not recommended for use in the synthesis of pharmaceuticals.

Cefdinir is primarily characterized by the presence of three reactive functional groups as follows:
a. The amino group attached to 2-position of the 2-(2-aminothiazol-4-yl)-2(Z)-oxyimino-acetamido function at the 7β-position;
b. The hydroxy group attached to the oxime function of the 2-(2-aminothiazol-4-yl)-2(Z)-oxyimino-acetamido function at the 7β-position; and
c. The carboxylic acid group at the 4-position, all of which are illustrated in the following structural diagram:

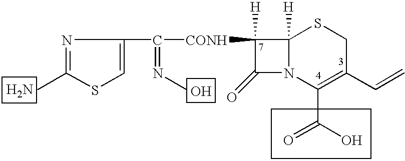

Side-products invariably form because of reactions between these functional groups with the reagents/chemicals employed for synthesis. Such side products are not only difficult to remove, but they also reduce yields, thereby increasing the cost of manufacture.

In addition, the oxime function in cefdinir, which is best disposed towards the syn or (Z)-configuration for the optimum biological activity is highly susceptible to isomerization to the undesired isomer having the anti or (E)-configuration. This anti or (E)-configuration invariably forms in varying proportions through employment of most of the prior art methods. This renders the product obtained therein, in many instances, unacceptable for pharmacoepial applications.

Taking the abovementioned limitations into account, and moreover, taking into consideration the therapeutic and commercial importance that cefdinir enjoys, there is an urgent need for a simple, convenient, non-hazardous, environmentally acceptable, and cost-effective method that overcomes the limitations of the prior art methods.

Such a need has been met by the present inventors, as would be evident from the discussion given hereinafter.

### SUMMARY OF THE INVENTION

The present invention relates to a crystalline form of cefdinir, which is stable and bioavailable.

The present invention also includes a simple, convenient, non-hazardous, environment friendly and cost-effective method for manufacture of a stable and bioavailable crystalline form of cefdinir.

Yet another object of the present invention is to provide an antimicrobial pharmaceutical composition comprising the stable, bioavailable crystalline form of cefdinir in admixture with pharmaceutically acceptable carriers for treatment of bacterial infections.

The present inventors have found that cefdinir of high purity and substantially free of isomers including the anti or (E)-isomer, can be obtained through an improved method utilizing a crystalline 2-tritylamino-(2-aminothiazol-4yl)-2-trityloxyimino acetic acid alkali metal salt dihydrate of formula (II), 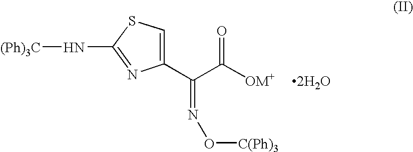
wherein M is an alkali metal salt, for acylation at the 7-position of a 7-amino-3-vinyl-3-cephem-4-carboxylic acid derivative of formula (III), 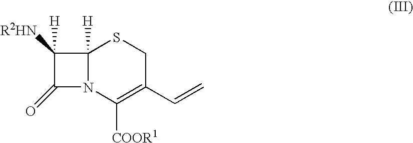
wherein R¹ is a trialkylsilyl group or a carboxylic acid protective group, and R² is a trialkyl silyl group or an organic sulfonic acid, in a suitable medium. Using a selective deprotection and isolation method, cefdinir can be isolated from the acylated compound obtained by the acylation. This cefdinir product has a well-defined crystalline structure in the form of a sesquihydrate, and exhibits excellent storage stability and bioavailability.

The use of crystalline 2-tritylamino-(2-aminothiazol-4yl)-2-trityloxyimino acetic acid alkali metal salt dihydrate of formula (II) as a compound for the synthesis of cefdinir offers several advantages. The crystalline 2-tritylamino-(2-aminothiazol-4yl)-2-trityloxyimino acetic acid alkali metal salt dihydrate (II) can be prepared by alkaline hydrolysis of the ester function of the corresponding carboxylic acid ester compound of formula (V).

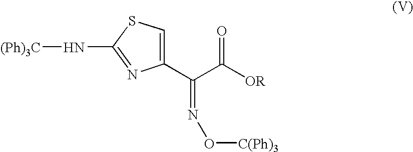

Under such conditions, the ester function of the corresponding carboxylic acid ester is selectively hydrolysed in the presence of an alkali metal hydroxide. This readily forms the alkali metal salt (II), leaving the two trityl groups in the molecule practically intact.

The alkali metal salt (II) obtained following this hydrolysis is a nice free flowing crystalline solid, having a water content of between about 5 to about 7%. This crystalline solid has been found to be relatively stable on storage, exhibiting practically no degradation. Of particular importance it has been found that hydrolysis of the acid sensitive trityl groups does not occur.

Next, the alkali metal salt (II) can be converted to the corresponding acid chloride or bromide by reaction with a suitable chlorinating/brominating agent. The acid halide thus formed, is reacted with a β-lactam compound (III) to synthesize cefdinir. This process provides for a substantial reduction in the formation of side products, such as the anti or (E) isomer, to produce a high yield of a high purity product.

The inventors have found that an additional step, in which the alkali metal salt (II) is hydrolyzed in the presence of an acid, to form a ditritylated carboxylic acid derivative (VI), 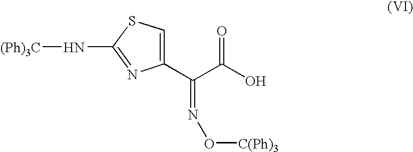
can also be employed. However, such hydrolysis results in partial removal of either one or both of the trityl protective groups in the molecule. In particular, the trityl protective group of the amino group attached to the aminothiazole ring is hydrolysed to an extent of 5-10%. The aminothiazole carboxylic acid compound (VI), unlike the alkali metal salt (II), is obtained essentially in the anhydrous form, as is evident from its water content of 0.5 to 0.7%. The aminothiazole carboxylic acid compound (VI), when converted to the acid chloride and reacted with the β-lactam compound (III), leads to the formation of substantial amounts of side-products and produces a very low yield of cefdinir, typically only about one third of that produced when the alkali metal salt (II) is used. Furthermore, when this additional acid hydrolysis step is employed the formation of the anti or (E)-isomer is higher. A comparison of the yield, quality and the level of impurities of cefdinir prepared by reaction of compounds (II) and (VI) with p-methoxybenzyl-7-amino-3-vinyl-3-cephem-4-carboxylate p-toluenesulfonic acid salt (compound III), respectively, is summarized in Table-I.

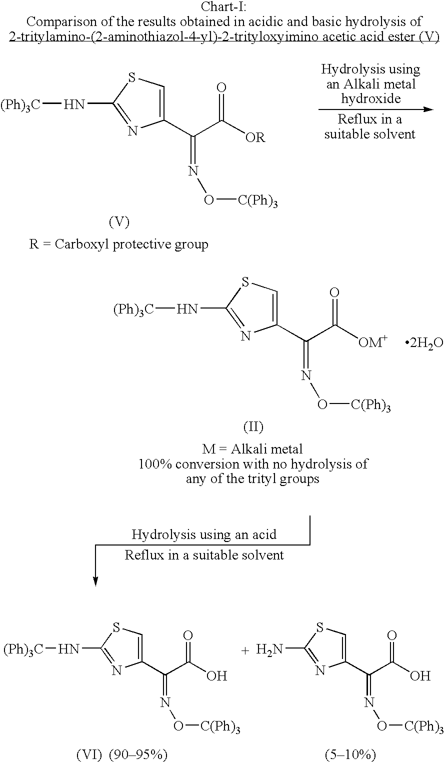

Further, the present inventors have found that this same result is achieved regardless of whether a silylated cephalosporin compound (III) or a 4-carboxylic acid ester compound (III) is used. In fact, when the acid chloride or the carboxylic acid derivative (VI) was used for reaction with the silylated cephalosporin compound (III), the conversion was only about 60%, with formation of impurities in the range of 15-20%. Thus, the purity and yield of the process when the additional step (acid hydrolysis of the alkali metal salt (II)) is employed is even worse when a silylated cephalosporin compound (III) is used in the acylation step.

All attempts to prepare the ditritylated carboxylic acid derivative (VI) through tritylation of the corresponding de-tritylated carboxylic acid analogue, i.e. 2-(2-aminothiazol-4-yl)-2-hydroxyimino acetic acid, were unsuccessful.

Thus, the advantage of using the alkali metal salt (II) for synthesis of cefdinir would be abundantly evident from the comparison given in Chart-I and Table-I, and the previous discussion.

In addition, the present inventors further found when a monotitylated aminothiazole acetic acid sodium salt of formula (VIII), wherein wherein M is an alkali metal salt, was used for coupling with the beta-lactam nucleus (III) the conversion did not proceed beyond 60% and levels of impurities between 15-20% formed.

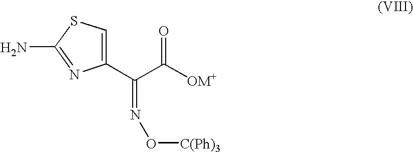

TABLE I
Comparison of the results obtained in preparation of cefdinir by reaction of the 2-tritylamino-
(2-aminothiazol-4yl)-2-trityloxyimino acetic acid alkali metal salt (II) and the 2-tritylamino-
(2-aminothiazol-4yl)-2-trityloxyimino acetic acid (VI) with p-methoxybenzyl-7-amino-3-
vinyl-3-cephem-4-carboxylate p-toluenesulfonic acid salt (corresponding to compound III)

| | | Synthesis using 2-tritylamino- | |
|---|---|---|---|
| | | (2-aminothiazol-4yl)-2- | Synthesis using 2-tritylamino- |
| | | trityloxyimino acetic acid alkali | (2-aminothiazol-4yl)-2- |
| No. | Details | metal salt (II) | trityloxyimino acetic acid (VI) |
| 01 | Synthesized through | Alkaline hydrolysis of the | Acidic hydrolysis of the alkali |
| | | corresponding carboxylic acid | metal salt (II) |
| | | ester compound (V) | |
| 02 | Purity of the compound | 98%, with hardly any hydrolysis | 85-90%, with 5-10% hydrolysis |
| | | of any of the trityl groups | of the trityl group attached to the |
| | | | amino group of the |
| | | | aminothiazole ring |
| 03 | Water content of the | - - - , essentially in the form of | 05 to 0.7%, essentially in the |
| | respective aminothiazole | a dihydrate | anhydrous form |
| | compounds obtained | | |
| 04 | Conversion to the | By reaction with POCl₃ in a | By reaction with POCl₃ in a |
| | corresponding acid chloride | suitable organic solvent in the | suitable organic solvent in the |
| | | presence of a base | presence of a base |
| 05 | Whether acid chloride isolated | No, formed in situ and reacted | No, formed in situ and reacted |
| | | with p-methoxybenzyl-7-amino- | with p-methoxybenzyl-7-amino- |
| | | 3-vinyl-3-cephem-4-carboxylate | 3-vinyl-3-cephem-4-carboxylate |
| | | p-toluenesulfonic acid salt in the | p-toluenesulfonic acid salt in the |
| | | same solvent | same solvent |
| 06 | % conversion to product on | 85-90% | 50-55% |
| | reaction with p- | | |
| | methoxybenzyl-7-amino-3- | | |
| | vinyl-3-cephem-4-carboxylate | | |
| | p-toluenesulfonic acid salt | | |
| 07 | % of p-methoxybenzyl-7- | Below Detectable Limits | 5-10% |
| | amino-3-vinyl-3-cephem-4- | | |
| | carboxylate p-toluenesulfonic | | |
| | acid salt unreacted | | |
| 07 | % side-products formed in the | None | About 15-25% |
| | reaction | | |
| 08 | Yield of cefdinir isolated | 30% w/w of p-methoxybenzyl-7- | 10% w/w of p-methoxybenzyl-7- |
| | | amino-3-vinyl-3-cephem-4- | amino-3-vinyl-3-cephem-4- |
| | | carboxylate p-toluenesulfonic | carboxylate p-toluenesulfonic |
| | | acid salt | acid salt |
| 09 | Purity of cefdinir isolated | ≧99.5% | About 98% |
| 10 | % of anti or (E)-isomer in | ≦0.2% | ≧0.5% |
| | finished product | | |

Thus, in addition to addressing the problems associated with the prior art methods, the process of the present invention is simple, convenient and cost-effective. Moreover, the process can be conducted so as to avoid the use of solvents frowned upon by environmentalists and which pose hazards in operability on a commercial scale.

Further, the process provides a highly stable and bioavailable form of cefdinir possessing a shelf life, dissolution rate, and bioavailability comparable to the currently marketed “Crystal A” form of cefdinir.

In one aspect, the present invention provides a stable, bioavailable crystalline form of cefdinir of formula (I), having a water content of between about 6.0 to about 7.0%, characterized by the following X-ray (powder) diffraction pattern

| d-spacing (Å) | Relative Intensity |
|---|---|
| 15.07 | 37.52 |
| 11.33 | 31.89 |
| 10.96 | 25.12 |
| 7.52 | 100.00 |
| 5.65 | 17.19 |
| 5.47 | 42.18 |
| 4.90 | 10.77 |
| 4.76 | 43.54 |
| 4.56 | 18.38 |
| 4.23 | 38.46 |
| 4.18 | 33.31 |
| 3.98 | 41.54 |
| 3.75 | 5.77 |
| 3.63 | 35.03 |
| 3.54 | 9.09 |
| 3.46 | 29.93 |
| 3.39 | 34.30 |
| 3.27 | 3.19 |
| 3.18 | 8.88 |
| 3.08 | 18.07 |
| 2.96 | 5.86 |
| 2.88 | 16.56 |
| 2.82 | 16.06 |
| 2.62 | 12.87 |
| 2.56 | 14.96 |
| 2.40 | 7.04 |
| 2.30 | 12.91 |
| 1.99 | 5.22 |
| 1.97 | 9.12 |

and IR spectrum values, (Nujol/KBr), cm⁻¹: 3297, 1781, 1666, 1190, 1134. It should be appreciated by those having skill in the art that the above diffraction pattern and IR spectrum are merely illustrative of the crystalline form of cefdinir comprising the present invention. Those of skill in the art will understand that any compound exhibiting a substantially similar diffraction pattern or IR spectrum falls within the scope the current invention, as set forth in the claims. For instance, a compound exhibiting a X-ray diffraction pattern having the above d-spacing values within about 5% would fall within the scope of the current invention.

In another aspect, the present invention includes an improved process for the preparation of the stable, bioavailable crystalline form of cefdinir of formula (I), having a water content of between about 6.0 to about 7.0%, and exhibiting the characteristic X-ray (powder) diffraction pattern and IR spectrum values described above.

One embodiment of the process comprises reaction of a crystalline compound of formula (II), 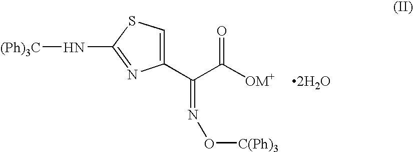
wherein M is an alkali metal, with a chlorinating or brominating agent in the presence of a water-immiscible organic solvent and also in the presence of an organic base to form the corresponding acid halide of formula (IV), 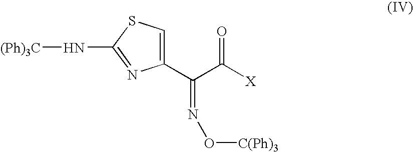
wherein X is Cl or Br.

Alternatively, the compound of formula (II), wherein M is an alkali metal, is reacted with a chlorinating agent in the presence of a dialkylamino pyridine, followed by an alkali metal bromide in the presence of a water-immiscible organic solvent and an organic base, to form the corresponding acid bromide of formula (IV), wherein X is Br.

The compound of formula IV, wherein X is chloride or bromide, is reacted in situ with a compound of formula (III), 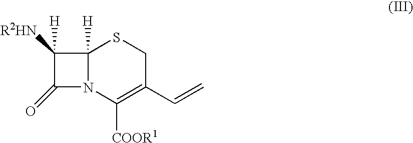
wherein R¹ is a trialkylsilyl group or a carboxylic acid protective group; and R² is a trialkyl silyl group or an organic sulfonic acid, to give a compound of formula (VII), 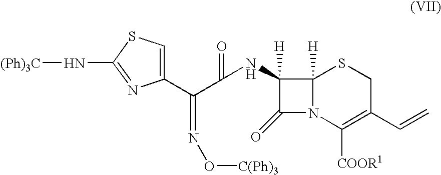
wherein R¹ is a trialkylsilyl group or a carboxylic acid protective group. The compound of formula VII can then be placed into solution, preferably without isolation, using a hydrocarbon solvent, and reacted with an acid to remove the protective groups, and produce crude cefdinir (I), either as the free base or as a salt with the acid used.

This crude product can then be crystallized by:
i) dissolving the crude cefdinir in water at a pH in the range of between about 6.3 to about 7.0, with the addition of an aqueous ammonia solution;
ii) optionally decolorizing the solution by treatment with activated carbon, and filtering off the carbon;
iii) bringing the pH of the clear solution to between about 2.3 to about 2.5 at a temperature of between about 0° C. to about 12° C. with the addition of acid to effect crystallization;
iv) agitating the crystals at a temperature of between about 0° C. to about 120° C. for a period of between about 30 minutes to about 120 minutes; and
v) filtering and drying the crystals to give the stable, bioavailable crystalline form of cefdinir (I). 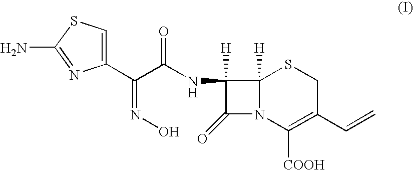
   This compound typically has a water content of between about 6.0 to about 7.0%, and exhibits the distinguishing features of the X-ray (powder) diffraction pattern and IR spectrum defined above.

In yet another aspect, the present invention provides an orally administrable antimicrobial pharmaceutical composition comprising the stable, bioavailable crystalline form of cefdinir of formula (I), having a water content of between about 6.0 to about 7.0%, generally characterized by the previously defined X-ray (powder) diffraction pattern and IR spectrum. This compound may be sold as an admixture with pharmaceutically acceptable carriers, and used to treat bacterial infections.

### BRIEF DESCRIPTION OF THE DRAWINGS

FIG. 1: The IR spectrum, recorded in KBr pellets, of the “crystalline form A” of cefdinir prepared under the method disclosed in U.S. Pat. No. 4,935,507.

FIG. 2: The IR spectrum, recorded in Nujol, of the “crystalline form A” of cefdinir prepared under the method disclosed in U.S. Pat. No. 4,935,507.

FIG. 3: The IR spectrum, recorded in KBr pellets, of the crystalline form of cefdinir prepared under the method of the present invention.

FIG. 4: The IR spectrum, recorded in Nujol, of the crystalline form of cefdinir prepared under the method of the present invention.

FIG. 5: The X-ray (powder) diffraction pattern of the “crystalline form A” of cefdinir prepared under the method disclosed in U.S. Pat. No. 4,935,507.

FIG. 6: The X-ray (powder) diffraction pattern of the crystalline form of cefdinir prepared under the method of the present invention.

### DETAILED DESCRIPTION OF THE PREFERRED EMBODIMENT

The various aspects of the present invention leading to production and characterization of the stable and bioavailable crystalline form of cefdinir are detailed below.

As previously mentioned, the present invention utilizes a crystalline 2-tritylamino-(2-aminothiazol-4yl)-2-trityloxyimino acetic acid alkali metal salt dihydrate of formula (II), 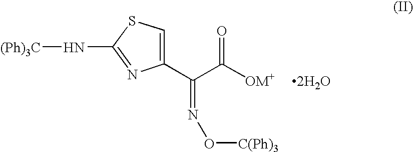
wherein M is an alkali metal salt, for acylation at the 7-position of a 7-amino-3-vinyl-3-cephem-4-carboxylic acid derivative of formula (III). 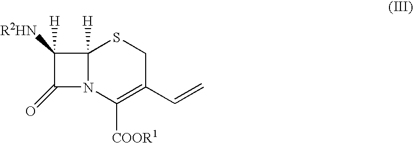
Preparation of the Alkali Metal Salt of Formula (II)

The alkali metal salt (II) can be prepared by refluxing a solution of the ester compound of formula (V), 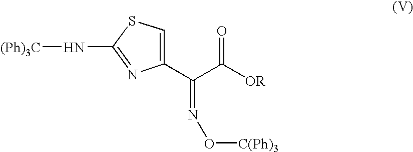
wherein R is an easily removable carboxyl protective group including a lower aliphatic alkyl group, in an organic solvent and in the presence of an aqueous solution of a base, such as alkali metal hydroxide or carbonate, for a period ranging between about 2.0 to about 3.0 hours.

Exemplary solvents that can be employed for the hydrolysis step include lower aliphatic alcohols such as methanol, ethanol, n-propanol, iso-propanol, n-butanol, iso-propanol and tertiary-butanol; aliphatic and cyclic ketones such as acetone, methyl ethyl ketone, methyl isobutyl ketone, cyclopentanone and cyclohexanone. Preferably the solvent is methyl ethyl ketone.

The alkali metal hydroxides or carbonates are typically employed as a concentrated solution in water. They can be employed in equimolar proportions to the ester compound (V), but more preferably 1.5 to 3.0 moles of the alkali metal hydroxides or carbonates are present for each mole of the ester compound (V).

Exemplary alkali metal hydroxides and carbonates include sodium hydroxide, potassium hydroxide, lithium hydroxide, sodium carbonate, potassium carbonate and lithium carbonate. Due to its low costs, the alkali metal hydroxide is preferably sodium hydroxide.

In a preferred method of the invention, a solution of sodium hydroxide in water is mixed with methyl ethyl ketone at a temperature between about 20° C. to about 30° C. The mixture is then further agitated at a temperature of between about 45° C. to about 50° C. for about 30 to about 45 minutes to obtain a homogeneous clear solution. Next, an ester compound of formula (V) is added to the solution at a temperature about between about 45° C. to about 50° C., and the resulting mixture is refluxed for about 2.0 to about 3.0 hours, or until completion of the hydrolysis as monitored by TLC or HPLC.

After the reaction is complete, water is added to the mixture, which is then cooled to a temperature of between about 0° C. to about 5° C. The mixture is then agitated at the same temperature for about 30 to about 60 minutes. The precipitated solid is collected by filtration, washed with methyl ethyl ketone, and dried to give the alkali metal salt (II), wherein M is sodium.

The crystalline sodium salt compound (II) exhibits the following physical, spectral, and solid state properties:

Water content: about 5 to about 7%, which corresponds to a dihydrate.

Mass Spectrum: 693 amu [corresponding to anhydrous salt (II)]

IR Spectrum (KBr, cm⁻¹): 3400, 1613, 1527

¹H NMR (DMSO-d₆, 6; 200 MHz): 6.42 (s, 1H); 7.12-7.29 (m, phenyl); 8.41 (9 s, 1H).

TGA thermogram: Weight loss at 100° C. (0.80%); 212° C. (5.4%); 227° C. (7.6%); 253° C. (16.7%); 258° C. (39.0%); and 336° C. (69.1%).

X-ray (powder) diffraction pattern:
(d, Å): 15.08, 11.18, 10.69, 7.92, 7.51, 7.14, 6.88, 6.66, 5.32, 5.00, 4.90, 4.41, 4.36, 4.30, 3.78, 3.72, 3.60, 3.50, 3.44; and
(2θ): 5.85, 7.89, 8.25, 11.14, 11.37, 12.37, 12.83, 13.26, 16.62, 17.72, 18.08, 20.11, 20.34, 20.59, 23.49, 23.86, 24.64, 25.41, 25.87).
   Again, these values are merely illustrative, and any compound exhibiting the distinguishing features of the above characteristic values can be used in the invention.

The starting ester compound (V) can be prepared by reaction of the corresponding 2-(2-aminothiazol-4-yl)-2-hydroxyimino acetic acid ester with trityl chloride in a suitable organic solvent as per the method described by R. Boucourt et. al. in Tetrahedron, 1978, 34, 2233-43.

Preparation of the Halide Salt of the Alkali Metal Salt of Formula (II)

The alkali metal salt (II) can then be converted to the corresponding acid chloride or bromide of formula (IV), wherein X is chlorine or bromine, by reaction with a chlorinating or brominating agent in a suitable water-immiscible organic solvent and in the presence of an organic base. The chlorinated or brominated product, preferably in situ (which has not been isolated), is then used in an acylation reaction with a 7-amino-3-vinyl-3-cephem-4-carboxylate compound (III) to yield a 7-[2-(2-tritylaminothiazoly-4-yl)-2-trixylhydroxyimino acetamido-3-vinyl-3-cephem-4-carboxylate of formula (VII).

Exemplary chlorinating agents for the formation of the acid chloride (formula IV, wherein X is Cl) include thionyl chloride, sulfury chloride, phosphorous trichloride, phosphorous pentachloride, phosphorous oxychloride, oxalyl chloride and the like. Suitable brominating agents that can be employed for formation of the acid bromide (formula IV, wherein X is Br) are selected from thionyl bromide, phosphorous tribromide, phosphorous pentabromide, and the like.

The chlorinating or brominating agent can be employed in equimolar proportions or in molar proportions in excess of the sodium salt compound (II). Chlorinating or brominating agents can be used in molar proportions in excess of the sodium salt compound (II), such as between about 1.5 to about 3.0 moles per mole of compound (II), and most preferably in the range of between about 1.3 to about 1.6 moles per mole of compound (II).

The acid halide formation reaction can be carried out in a solvent, which has limited miscibility with water. As used throughout this specification, a compound which has limited miscibility with water refers to an organic solvent which that has no, or very limited, miscibility with water. Such solvents include, but are not limited to, chlorinated hydrocarbons, such as dichloromethane or dichloroethane and the like; or aromatic hydrocarbons such as benzene, toluene, xylene, and the like.

The acid halide formation reaction can be carried out in the presence of an organic base to trap the liberated hydrogen chloride or hydrogen bromide. Suitable organic bases to perform this step include alkyl amines, such as dimethylamine, diethylamine, trimethylamine, triethylamine, triisopropylamine and tertiarybutylamine and the like; dialkylamines such as dimethylaniline and diethylaniline; pyridine; dicyclohexylamine; DBN, DBU, N-methylmorpholine, and the like. The preferred organic base, however, is pyridine.

The organic base can be used in equimolar proportions or in molar proportions in excess of the sodium salt compound (II). Preferably, the organic base is employed in molar proportions in excess of the sodium salt compound (II). More preferably the organic base is present in a ratio of about 1.0 to about 3.0 moles per mole of compound (II), and most preferably in a ration of about 1.2 to about 2.0 moles per mole of compound (II).

The acid halide formation reaction can be carried out from very low temperatures of about 65° C. to higher temperatures of about −10° C. However, it is preferable to carry out the reaction at a temperature of between about −35° C. to about −25° C.

Alternatively, the acid bromide compound, i.e. the compound of formula (IV) wherein X is Br, can be prepared by reaction of the sodium salt (II) with any of the chlorinating agents mentioned hereinbefore, in the presence of a dialkylaminopyridine and an alkali metal bromide in a water-immiscible organic solvent and in the presence of an organic base, under the conditions in which the chlorine atom of the chlorinating agent is substituted by a bromine atom, to produce an acid bromide of formula (IV).

The dialkylaminopyridine can comprise dimethylaminopyridine or diethylaminopyridine, while the alkali metal bromide cam be sodium bromide, potassium bromide, lithium bromide, and the like. Of these, dimethylaminopyridine and sodium bromide are preferred.

The dialkylaminopyridine can be used in equimolar proportions or in molar proportions in excess of the chlorinating agent used. Preferably, the dialkylaminopyridine is present in a ratio of about 1.0 to about 3.0 moles per mole of the chlorinating agent and most preferably it is present in a ratio of about 1.2 to about 2.0 moles per mole of the chlorinating agent employed.

Similarly, the alkali metal bromide is used in equimolar proportions or in molar proportions in excess of the chlorinating agent used. Preferably, the alkali metal bromide is present in a ratio of about 1.0 to about 3.0 moles per mole of the chlorinating agent. More preferably, the alkali metal bromide is present in a ratio of about 1.2 to about 2.0 moles per mole of the chlorinating agent used.

Preparation of Crude Cefdinir

The acid halide compound (IV) thus prepared, 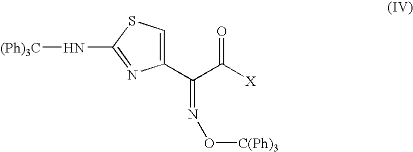
wherein X is Cl or Br, can be reacted, preferably in situ without isolation, with the compound of formula (III), 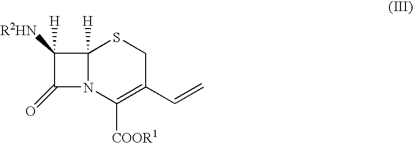
wherein R¹ is a trialkylsilyl protective group or a carboxylic acid protective group, and R² is a trialkyl silyl group or a organic sulfonic acid protective, to produce a compound of formula (VII), 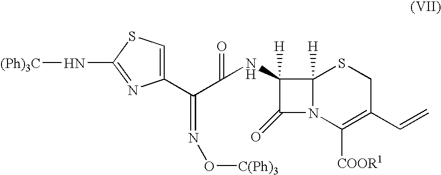
wherein R¹ is a trialkylsilyl protective group or a carboxylic acid protective group.

The carboxylic acid protective group, R¹ can be one that is conventionally utilized in chemical synthesis and known to those of skill in the art. However, with a view to the two trityl groups in the end product (VII) of acylation, it is advantageous to use a protective group that could be removed by acidic hydrolysis along with the two trityl protective groups. Therefore, it is highly advantageous to use protective groups like p-methoxybenzyl or benzhydryl, which, along with the two trityl groups in the molecule, can be easily removed in one operation by treatment with an acid, such as, trifluoroacetic acid.

When the group R¹ is a carboxylic acid protective group, especially the p-methoxybenzyl or benzhydryl groups previously mentioned, the group R² is normally an acid addition salt of the 7-amino function of compound (III), preferably acid addition salts with organic sulfonic acids like methanesulfonic or p-toluenesulfonic acid. The p-methoxybenzyl/benzhydryl 7-amino-3-vinyl-3-cephem-4-carboxylate p-toluenesulfonate and methanesulfonate salts corresponding to the compound of formula (III) can be prepared by known methods, preferably through the methods disclosed in U.S. Pat. No. 3,994,884.

Alternatively, the groups R¹ and R² in compound of formula (III) can be trialkylsilyl groups. Such silylated compounds (III) can be prepared by methods known in the art. For instance, these compounds can be prepared by reacting 7-amino-3-vinyl-3-cephem-4-carboxylic acid of formula (IX) 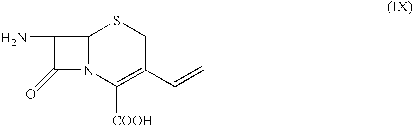
with a silylating agent. Appropriate silylating agents include silylated amides, such as N,O-bis-(trimethylsilyl) acetamide (BSA) or Bis-silyl urea (BSU), or a mixture of hexadimethylsilazane (HMDS) and trimethylchlorosilane (TMCS).

The silylation can be effected by reaction of 7-amino-3-vinyl-3-cephem-4-carboxylic acid (IX) with the silylating agent in a water-immiscible organic solvent, such as chlorinated hydrocarbons like dichloromethane, acetic acid (C₁₋₄) alkyl esters like ethyl acetate, ethers like diisopropyl ether, and the like. Chlorinated hydrocarbons are preferred and amongst these dichloromethane is the most preferred.

Typically, the 7-amino-3-vinyl-3-cephem-4-carboxylic acid (IX) is dissolved in dichloromethane and water is azeotropically removed from the solution. To this solution is added the silylating agent. The mixture is then heated or refluxed until the silylation is complete. The solution of the silylated compound (III) thus obtained is cooled to about −40° C. to about −60° C. under an inert gas atmosphere for reaction with the sodium salt (II).

The acylation reaction of compound (III) with the acid halide (IV) can be carried out in the same water-immiscible organic solvent as used in the silylation of compound (III) at a temperature of between about −65° C. to about −100° C. However, it is preferable to carry out the reaction at a temperature of between about −35° C. to about −25° C. The acylation reaction is normally complete in about 1 to about 2 hours and gives conversion to compound (VII) in greater than about 95%.

In one embodiment, the crystalline 2-tritylamino-(2-aminothiazol-4yl)-2-trityloxyimino acetic acid alkali metal salt dihydrate of formula (II) is dissolved in a water-immiscible organic solvent and excess moisture is removed by azeotropic distillation. The solution is cooled to a temperature of between about −20° C. to about −25° C. to which is added the p-methoxybenzyl/benzhydryl 7-amino-3-vinyl-3-cephem-4-carboxylate p-toluenesulfonate/methanesulfonate salt or the (N,O)-bis silylated 7-amino-3-vinyl-3-cephem-4-carboxylate, corresponding to a compound of formula (III) and the mixture agitated at the same temperature for about 10 to about 15 minutes. An organic base is then added to the mixture which is again agitated at the same temperature for an additional 10 to about 15 minutes. Slowly, over a period of about 30 to about 45 minutes, a solution of the chlorinating agent is added in the same water-immiscible organic solvent. During this addition, the temperature is maintained between about −20° C. to about −25° C. and thereafter the reaction is agitated until completion of the reaction.

In another embodiment, the crystalline 2-tritylamino-(2-aminothiazol-4yl)-2-trityloxyimino acetic acid alkali metal salt dihydrate of formula (II) can be dissolved in the water-immiscible organic solvent and excess moisture removed by azeotropic distillation. The solution can then be cooled to a temperature of between about −200° C. to about −25° C. to which a p-methoxybenzyl/benzhydryl 7-amino-3-vinyl-3-cephem-4-carboxylate p-toluenesulfonate/methanesulfonate salt or a (N,O)-bis silylated 7-amino-3-vinyl-3-cephem-4-carboxylate, corresponding to a compound of formula (III), is added. The resulting mixture is agitated at the same temperature for about 10 to about 15 minutes. The organic base is then added to the mixture and again agitated at the same temperature for a further 10 to about 15 minutes. To the resulting solution is successively added a solution of the chlorinating agent, the dialkylaminopyridine, and the alkali metal bromide, and the reaction mixture is agitated at a temperature of between about −20° C. to about −25° C. until the completion of the reaction.

In both the abovementioned methods, after the acylation reaction has reached completion the pH of the reaction mixture can be adjusted to between about 2.5 to about 3.0 by addition of a base, such as aqueous sodium hydroxide. The temperature can then be raised to room temperature. The water-miscible organic solvent is then evaporated to reduce the mixture to a low volume whereupon the reaction mixture is diluted with an aromatic hydrocarbon such as benzene, toluene, xylene or the like. The reaction mixture can once again be concentrated down to a low volume and diluted with water. The organic layer is separated from the aqueous phase and evaporation of the solvent yields compound (VII) as a free flowing solid, which can be used for the next deprotection step.

However, it is advantageous to use the solution of compound (VII) in the hydrocarbon solvent, without isolation, for deprotection of the protective groups.

A p-methoxybenzyl/benzhydryl 7-amino-3-vinyl-3-cephem-4-carboxylate p-toluenesulfonate/methanesulfonate salt, corresponding to a compound of formula (III), can be used for an acylation reaction with the sodium salt of formula (II). The two trityl protective groups, as well as the p-methoxybenzyl/benzhydryl protective groups, can be removed in one step using a strong acid, like trifluoroacetic acid, to afford cefdinir (I). When (N,O)-bis silylated 7-amino-3-vinyl-3-cephem-4-carboxylate, corresponding to a compound of formula (III) is used for the acylation reaction with the sodium salt of formula (II), the two trityl protective groups can be removed using any organic or inorganic acid. For instance, methanesulfonic acid, hydrochloric acid, formic acid, and the like, would be acceptable acids.

The hydrolysis of the respective protective groups is typically effected by slowly adding the desired acid to the solution of compound (VII) in an aromatic hydrocarbon at a temperature of between about 0° C. to about 20° C. with agitation for a period of between 1 to 3 hours until completion of the reaction. The cefdinir (I), thus obtained can be isolated by precipitation through the addition of water, and collected by filtration.

Crystallization of the Crude Cefdinir

The crude cefdinir thus obtained can be dried, but preferably the wet material is used for purification. Under this method, the crude material can be suspended in water and the mixture cooled to a temperature between about 0° C. to about 12° C. Through the slow addition of aqueous ammonium hydroxide, the pH of the suspension can be adjusted to between about 6.3 to about 7.0, wherein a clear solution results. The solution can be charcoalized, if necessary, wherein the pH of the filtrate is slowly adjusted to between about 2.3 to about 2.5 by the addition of a mineral acid. Crystallization is effected by agitation of the solution at the same temperature (about 0° C. to about 12° C.) for a period of about 1 to about 3 hours. Thereafter, the crystals can be collected by filtration and dried, thereby producing cefdinir (I) with a purity greater than about 99.5% and substantially free from impurities, especially the (E) or anti-isomer.

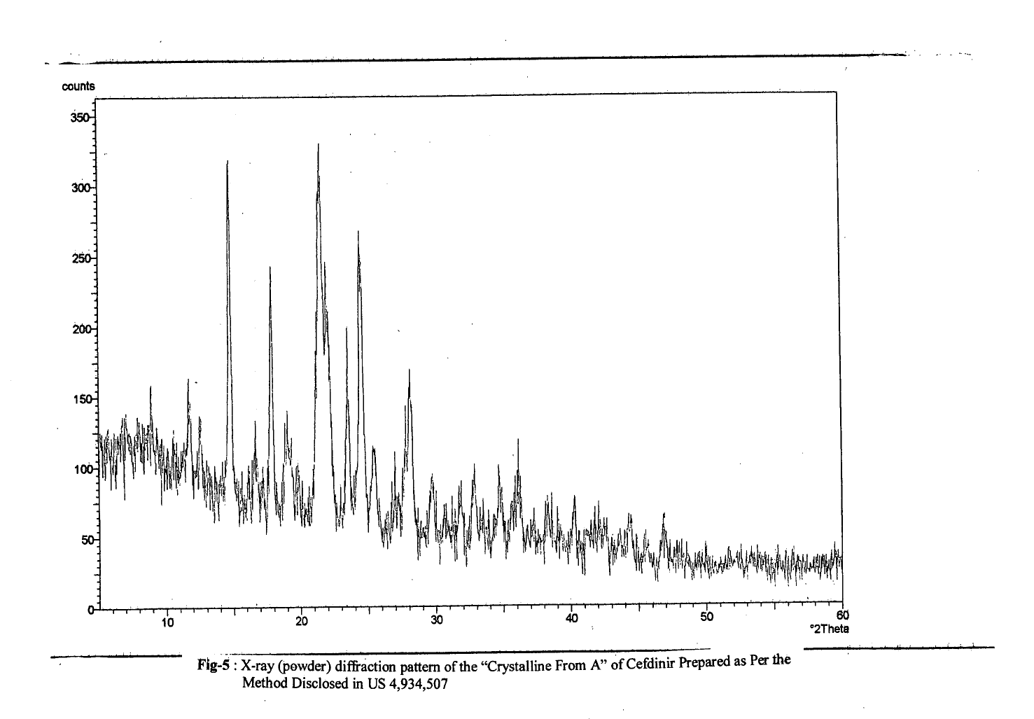

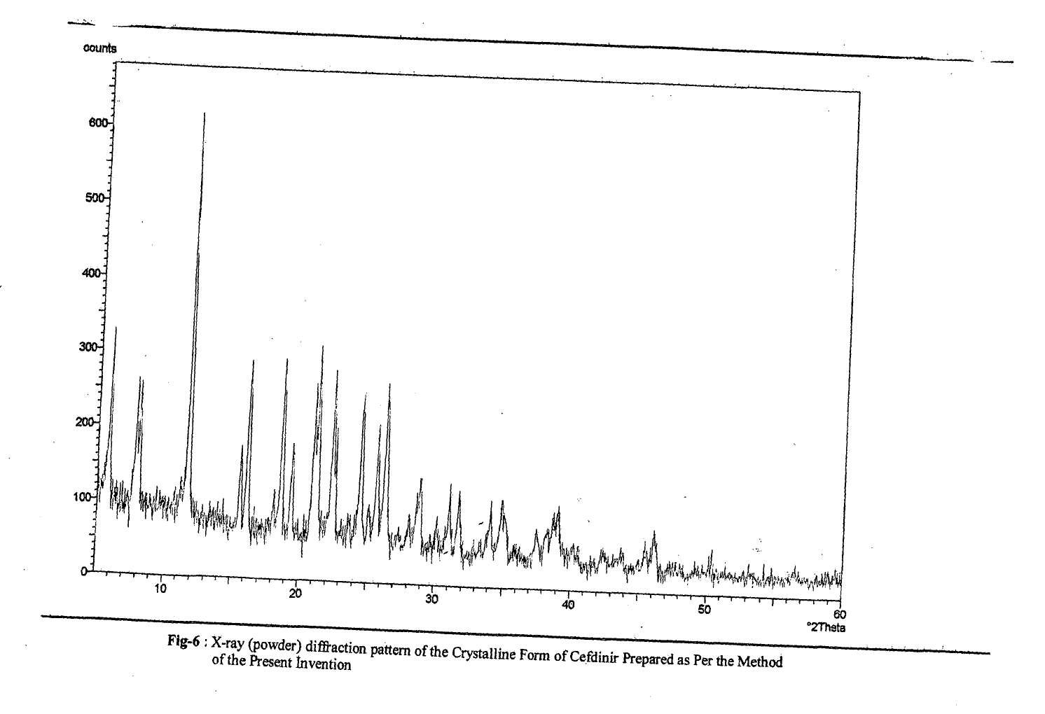

The cefdinir thus obtained exhibits the X-ray (powder) diffraction pattern summarized in Table-II and depicted in FIG. 6, which is distinctly different from that of the “crystalline form A” disclosed in U.S. Pat. No. 4,935,507 and depicted in FIG. 5.

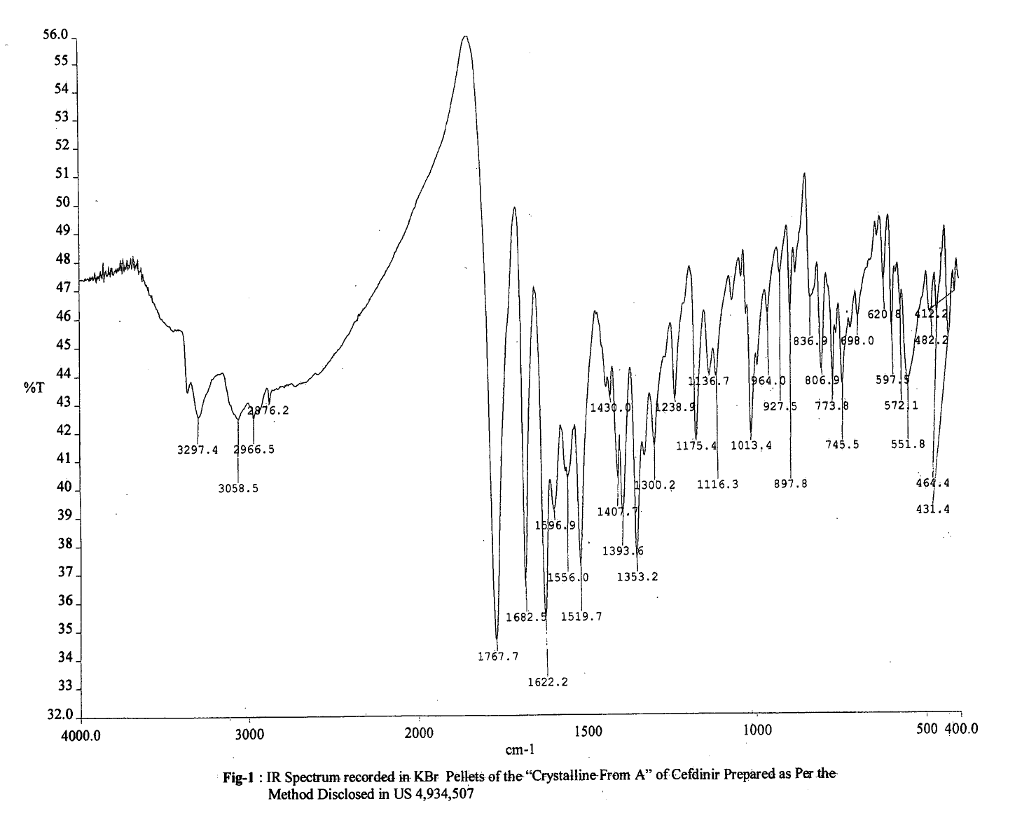

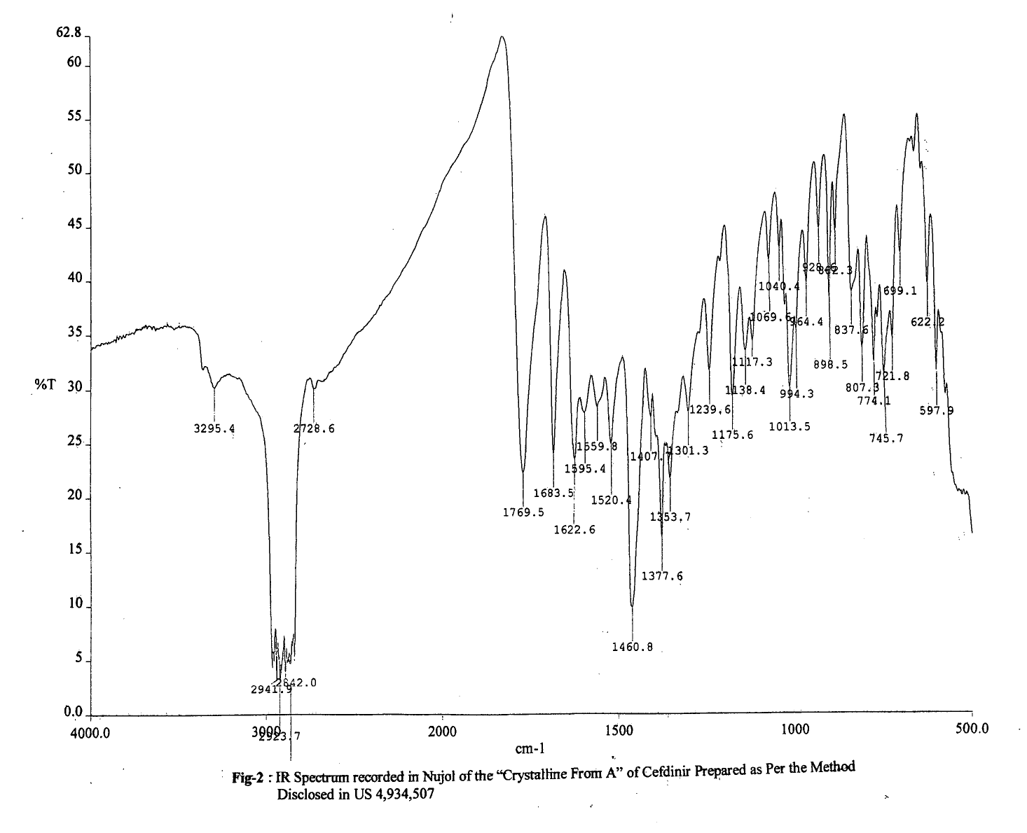

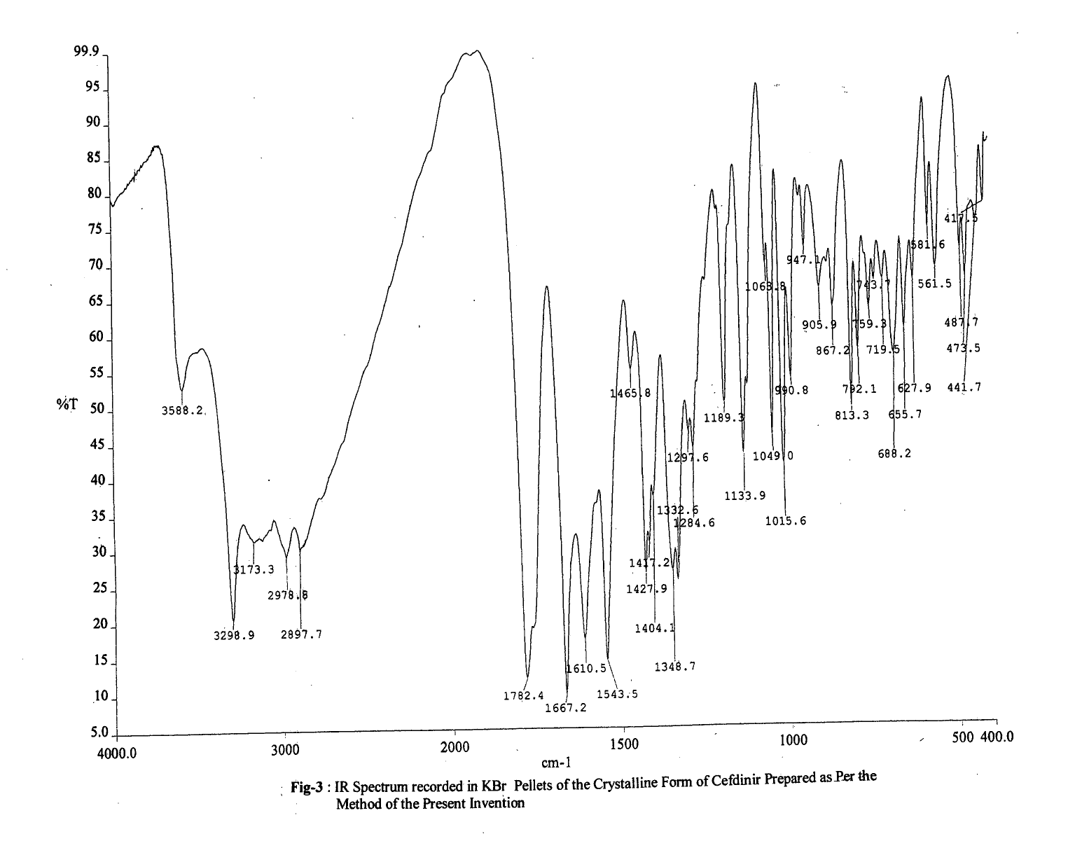

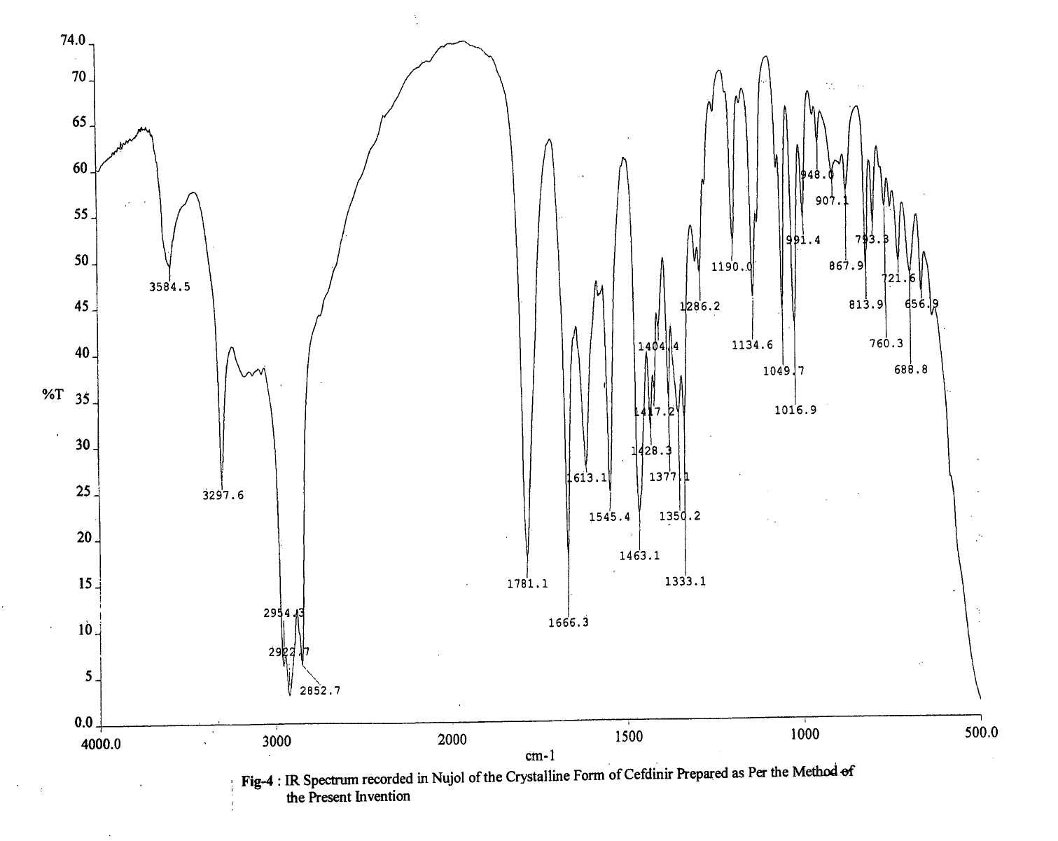

Further, the IR spectrum of the cefdinir obtained by the present invention, recorded in both KBr and Nujol, viz. 3297, 1781, 1666, 1190, 1134 cm⁻¹ and depicted in FIGS. 3 and 4 respectively, are also distinctly different from the IR spectrum of the “crystalline form A” of cefdinir, which has been recorded in both KBr and Nujol, viz. 1760, 1670, 1620 cm⁻¹ and which is depicted in FIGS. 1 and 2 respectively.

The pure cefdinir (I) obtained by the process of the present invention exhibits excellent thermal stability at 40° C. (75% Rh) with very little drop in potency, as can be seen from the Accelerated Storage Stability Data provided in Table-III.

Further, the dissolution rate of the crystalline cefdinir of the present invention is found to be superior to not only that of the reference “Crystal A” but also to the material prepared under the method disclosed in U.S. Application No. 2003/0204082 A (Dobfar et al.), as exemplified in Table-IV.

In addition, the process for preparation of the crystalline cefdinir is simple, inexpensive and convenient compared to the prior art methods. Under the present invention, cefdinir can be prepared in one-pot, without requiring the isolation of any intermediate compounds at any stage. Most importantly, cefdinir is isolated from water and, unlike the prior art methods, it can be isolated from aqueous organic solvents. This considerably reduces both hazards in operability and waste disposal problems.

TABLE II
Characteristic X-ray (powder) diffraction pattern of cefdinir obtained
by the present invention (exemplary only).

| d-spacing (Å) | Relative Intensity |
|---|---|
| 15.07 | 37.52 |
| 11.33 | 31.89 |
| 10.96 | 25.12 |
| 7.52 | 100.00 |
| 5.65 | 17.19 |
| 5.47 | 42.18 |
| 4.90 | 10.77 |
| 4.76 | 43.54 |
| 4.56 | 18.38 |
| 4.23 | 38.46 |
| 4.18 | 33.31 |
| 3.98 | 41.54 |
| 3.75 | 5.77 |
| 3.63 | 35.03 |
| 3.54 | 9.09 |
| 3.46 | 29.93 |
| 3.39 | 34.30 |
| 3.27 | 3.19 |
| 3.18 | 8.88 |
| 3.08 | 18.07 |
| 2.96 | 5.86 |
| 2.88 | 16.56 |
| 2.82 | 16.06 |
| 2.62 | 12.87 |
| 2.56 | 14.96 |
| 2.40 | 7.04 |
| 2.30 | 12.91 |
| 1.99 | 5.22 |
| 1.97 | 9.12 |

TABLE III
Accelerated Storage Stability Data of Cefdinir Prepared by the Method
of the Present Invention packed in Aluminum pouch and kept
at 40 ± 2° C. at Rh 75 ± 5%

| | | After | After | After |
|---|---|---|---|---|
| Details | Initial | 30 days | 60 days | 180 days |
| Water Content (%) | 7.05 | 7.46 | 7.27 | 7.60 |
| pH (1% Aqueous | 3.40 | 3.46 | 3.45 | 3.55 |
| suspension) | | | | |
| Assay by HPLC | 98.37 | 97.92 | 97.38 | 96.65 |
| (% on Dry Basis) | | | | |

TABLE IV
Comparison of The Dissolution Rate of cefdinir prepared as per the method of the present
invention with that of the reference “Crystal A” and the cefdinir prepared as per the
method of Dobfar's US Application No. 2003/0204082 A1in 0.07 N Hydrochloric acid.

| | Dissolution Profile in mg/cm² | | |
|---|---|---|---|
| | Cefdinir Prepared as per | “Crystal A” form of | Cefdinir Prepared as per |
| Time | the Method of Present | Cefdinir Prepared as per | the Method of Dobfar's US |
| In | Invention | the Method of U.S. | Application No. |
| Mins | Batch # 01 | Pat. No. 4,935,507 | 2003/0204082 A1 |
| 15 | 4.15 | 2.31 | 4.59 |
| 30 | 8.26 | 3.40 | 6.01 |
| 45 | 10.31 | 4.40 | 8.70 |
| 60 | 14.66 | 5.16 | 10.42 |
| 75 | 16.29 | 6.32 | 12.23 |
| 90 | 19.47 | 7.23 | 14.94 |
| 105 | 22.17 | 7.86 | 16.79 |
| 120 | 23.01 | 8.57 | 19.02 |
| IDR | 0.1831 | 0.0601 | 0.1392 |

IDR: Intrinsic Dissolution Rate in mg/cm²/min

Formulations Involving the Crystalline Form of Cefdinir of the Present Invention

Further, it has been found that the crystalline cefdinir can be successfully formulated into oral dosage forms like capsules, tablets and suspensions, which not only exhibit excellent storage stability but also are bioavailable, thereby providing a pharmaceutical composition of cefdinir, which can be used for the effective treatment of bacterial infections. This form of cefdinir is suitable for human consumption.

Dosage forms for oral administration can be made into different forms such as capsules, tablets, suspensions, and the like, of various strengths of the active ingredient in admixture with pharmaceutically acceptable carriers.

Capsules are generally preferred because they are tasteless, essentially innocuous, easily administered, easily filled extemporaneously, and can be manufactured in large numbers. Additionally, it is easier to swallow capsules than other dosage forms, such as a solid tablet. Further, many individuals have difficulty in swallowing a solid dosage form. In view of this, a liquid dosage form, such as a suspension, meets the requirement.

As previously discussed, it is highly advantageous to use the crystalline cefdinir of the present invention as the active ingredient for formulation both into a capsule or a suspension form. Due to its inherent stability and bioavailability, cefdinir of the present invention is capable of imparting the same stability and bioavailability without much loss in potency when formulated into capsules and/or suspensions.

As used herein, a stable dosage form means a formulation, such as a capsule or suspension, that is capable of remaining in a pharmaceutically acceptable condition for a prolonged period of time. Preferably, a stable dosage remains in a pharmaceutically acceptable condition for at least six months, more preferably at least a year, and most preferably more than three years. With respect to a suspension, a pharmaceutically acceptable condition means that there is little loss in potency of the drug, there is no significant crystal growth, and no sediments form which require greater than minor agitation to be re-suspended. In other words, a pharmaceutically acceptable condition means the suspension is free sediments that are not readily re-suspendable.

The active ingredient, i.e. the crystalline cefdinir of the present invention, should be present in an amount sufficient to obtain the desired benefits of the compound. Preferably the active ingredient is present in an amount from about 30% to about 95% by weight of the average fill weight of the capsule or the suspension.

The active ingredient can be formulated as an admixture with pharmaceutically acceptable carriers. Typical carriers that can be employed include a disintegrant and a lubricant. Disintegrants and lubricants are well known in the pharmaceutical sciences. Suitable disintegrants include starch, croscarmellose sodium, crospovidone, sodium starch glycolate, croscarmellose calcium, microcrystalline cellulose and polacralin potassium, and the like. Suitable lubricants include magnesium stearate, sodium stearyl fumarate, hydrogenated vegetable oil, hydrogenated castor oil, hydrogenated cottonseed oil, stearic acid and calcium stearate, colloidal silicon dioxide and the like.

The disintegrant and lubricant are selected such that they provide an effective amount of the disintegrant and/or an effective lubricating amount of the lubricant, respectively. For example, a typical formulation can contain from about 0% to about 30% by weight of a disintegrant and about 0% to about 10% percent by weight of a lubricant. In a preferred embodiment, the formulation contains from about 1% to about 10% by weight of a disintegrant and about 0.2% to about 2% percent by weight of a lubricant.

The pharmaceutical composition can be manufactured by methods known in the art. However, the release characteristics of the composition depends on the method chosen for manufacture.

With respect to the capsule formulation, in order to obtain the optimum release characteristics of the drug, the active ingredient and the disintegrant, preferably crosscarmellose calcium, can be mixed together with the lubricant/glidants, preferably colloidal silicon dioxide and magnesium stearate, wherein the blend is granulated by compaction followed by sieving. The granules obtained are lubricated further and filled into the empty hard gelatin capsule shells.

With regard to the suspension form, the active ingredient, in dry powder form, is typically admixed with water, which acts as the carrier, to form a suspension for oral dosage. The suspension thus produced is surprisingly stable; it has been found to be free from any settling for at least two weeks.

In addition, the composition can contain other additives, such as a suspending agents, thickening agents, preservatives, pH modifiers, bulking agents, flavouring agents, and the like. Any other desirable ingredients can be included provided such ingredients do not have a deleterious effect on the activity of cefdinir. Such compounds are well known to those of skill in the art.

Examples of suitable suspending agents include xanthan gum, hydroxypropylmethylcellulose, methylcellulose, carageenan, sodium carboxymethyl cellulose, and sodium carboxymethyl cellulose/microcrystalline cellulose mixes, particularly sodium carboxymethyl cellulose/microcrystalline cellulose mixtures.

Suitable suspending agents further include thixotropic suspending agents such as xanthan gum, carageenan and sodium carboxymethyl cellulose/microcrystalline cellulose mixtures and mixtures thereof. More preferred of these are xanthan gum and gaur gum.

One thickening agent found suitable for the present formulation is silicon dioxide, although others can also be used.

Preservatives may be incorporated into the formulation. A few water soluble preservatives found useful in present invention include sodium benzoate, sodium citrate, and benzalkonium chloride. When a preservative is used, it is preferably sodium benzoate.

Sweeteners that can be used, among others, include sugars, such as fructose, sucrose, glucose, maltose, or lactose; and non-calorie sweeteners such as aspartame. Aspartame can be used alone or in combinations with another non-caloric or low caloric sweetener, especially those which have been shown to have a synergistic sweetening effect with aspartame, such as saccharin, acesulfame, thaumatin, chalcone, cyclamate, stevioside, and the like. These sweetener compositions are more economical and impart good sweetness without any undesirable after-taste.

Bulking agents can be included to provide structure and mouth-feel qualities, which are normally provided by sucrose, fructose, sorbitol, or in the case of non-dairy desserts, vegetable, animal fat, or honey. Thus, sucrose can acts both as a sweetener and as a bulking agent.

The formulation can also include pH modifiers. Some pH modifiers which have been found useful in the formulation include sodium citrate, citric acid, tartaric acid, malic acid, sodium bicarbonates, sodium carbonate, and the like.

A capsule containing a Label claim of 300 mg/capsule including crystalline cefdinir of the present invention as the active ingredient admixed with a pharmaceutically active carrier(s), can be prepared with the composition summarized in Chart-II.

CHART II
A typical capsule composition (EG Capsules, size ‘0”) containing a Label
Claim (300 mg/Capsule) comprising the crystalline cefdinir of the present
invention

| | Unit Quantity |
|---|---|
| Ingredient | (mg/Capsule) |
| The crystalline Cefdinir of | 300.00 |
| the Present Invention | |
| Carboxymethyl Cellulose | 70.50 |
| Calcium USNF | |
| Colloidal Silicon Dioxide USNF | 1.50 |
| Magnesium Stearate USNF | 3.00 |
| Total Fill Weight | 375.00 |

A typical oral suspension containing a Label claim of 125 mg/5 ml including crystalline cefdinir of the present invention as the active ingredient admixed with a pharmaceutically active carrier(s), can be prepared with the composition summarized in Chart-II.III.

CHART III
A typical Suspension Composition containing a Label Claim
(125 mg/5 ml) comprising the crystalline cefdinir of the present invention

| | Unit Quantity |
|---|---|
| Ingredient | (mg/5 ml) |
| The crystalline Cefdinir of | 125.00 |
| the Present Invention | |
| Xanthan Gum USNF | 3.75 |
| Guar GumUSNF | 10.00 |
| Sodium Benzoate USNF | 10.00 |
| Sodium Citrate USP | 9.00 |
| Citric Acid USP | 5.00 |
| Strawberry Flavour | 25.00 |
| Colloidal Silicon Dioxide USNF | 15.00 |
| Sucrose USNF | 3047.25 |
| Total Weight | 3250.00 |

Both the abovementioned capsule form and the suspension form are found to be stable on storage as illustrated by the stability data provided in Chart-IV.

CHART IV
Stability data of the pharmaceutical composition (Capsule and Suspension)
comprising the crystalline cefdinir of the present invention

| | | Assay (%) after 3 |
|---|---|---|
| | | Months at 40° C. |
| Type of Composition | Initial Assay (%) | at 75% Rh |
| Capsule, corresponding to | 101.66 | 100.51 |
| composition given in Chart-II | | |
| Suspension, corresponding to the | 101.14 | 91.38 |
| composition given in Chart-III | | |

Bioequivalence studies comparing both the capsule and suspension form of the cefdinir of the present invention with the marketed Omnicef® Capsules and Suspensions prove that the crystalline cefdinir of the present invention is bioavailable and the bioavailability is equivalent to that of the marketed Omnicef® Capsules and Suspensions.

The bioequivalence study results carried out with both capsules and suspensions are summarized in Chart-V and VI respectively.

CHART V
The Bioequivalence Study Results carried out on Capsules containing the
crystalline cefdinir of the present invention compared with the marketed
Omnicef ® Capsule of M/S Abbott Laboratories as Reference Standard

| | Capsule containing the | | |
|---|---|---|---|
| | crystalline cefdinir of | | |
| | the present invention | | |
| | conforming to the | | |
| | composition | Omnicef ® Capsules | |
| | summarized in Chart- | marketed by M/S | Ratio |
| Study | II | Abbott Laboratories | (Test/ |
| Parameter | (Test) | (Reference) | Reference) |
| Cₘₐₓ | 1.412 | 1.212 | 116.50 |
| (ng/ml) | | | |
| AUC 0-t | 6.954 | 6.194 | 112.30 |
| (ng/h/ml) | | | |
| AUC 0-inf | 7.089 | 6.378 | 111.20 |
| (mcg/h/ml) | | | |

CHART VI
The Bioequivalence Study Results carried out on Suspensions containing
the crystalline cefdinir of the present invention compared with the
marketed Omnicef ® Suspension of M/S Abbott Laboratories
as Reference Standard

| | Suspension containing | | |
|---|---|---|---|
| | the crystalline cefdinir | | |
| | of the present invention | | |
| | conforming to the | Omnicef ® | |
| | composition | Suspension | |
| | summarized in Chart- | marketed by M/S | Ratio |
| Study | III | Abbott Laboratories | (Test/ |
| Parameter | (Test) | (Reference) | Reference) |
| Cₘₐₓ | 2.616 | 2.573 | 101.70 |
| (ng/ml) | | | |
| AUC 0-t | 13.510 | 13.191 | 102.40 |
| (ng/h/ml) | | | |
| AUC 0-inf | 13.803 | 13.487 | 102.30 |
| (mcg/h/ml) | | | |

The invention is further described by the following examples, which should not be construed as to limiting the scope of the invention, which is defined only by the claims.

### REFERENCE EXAMPLES

### Preparation of (Z)-(Tritylamino-2-thiazol-4yl)-2-Tritylxoyimino ethyl acetate (V)

To a suspension of (Z) 2-(2-aminothiazol-4-yl)-2hydroxyimino ethyl acetate (80 g, 0.372 moles) in acetonitrile (840 ml) was added to triethylamine (155.6 ml, 1.11 moles) and the mixture was stirred at a temperature of between 20° C. to 25° C. for 10 minutes, under one atmosphere of nitrogen. To this was added trityl chloride (238.6 g, 0.858 moles) in one lot under gentle stirring. Thereafter, the reaction was stirred at a temperature of between 55° C. to 60° C. for 1 hour. Acetonitrile was distilled out at atmospheric pressure and to the resulting thick slurry was added water (2400 ml). A precipitate formed which was filtered, washed with water, and dried to give 259 g (99.2%) of (Z)-(Tritylamino-2-thiazol-4yl)-2-Tritylxoyimino ethyl acetate (V) as a white solid.

### Example-I

A solution of sodium hydroxide (11.47 g, 0.286 moles) in water (12 ml) was mixed with methyl ethyl ketone (800 ml) to form a clear solution. The solution was maintained at a temperature of between 45° C. to 50° C. and (Z)-(Tritylamino-2-thiazol-4yl)-2-Tritylxoyimino ethyl acetate (V, 100 g, 0.143 moles) was added in one lot. The reaction mixture was refluxed for 2.25 hours and then water (200 ml) was added under stirring. The mixture was cooled to a temperature of between 0° C. to 5° C. under stirring and the precipitated solid filtered, washed with methyl ethyl ketone and dried under vacuum to give 90 g (90.82%) of (Z)-(Tritylamino-2-thiazol-4yl)-2-Tritylxoyimino acetic acid sodium salt (compound II) having a purity of 97%. The cefdinir product was analyzed and the following results were obtained:

Water content: between 5 to 7%

Mass Spectrum: 693 amu (corresponding to anhydrous salt (II)

IR Spectrum (KBr, cm⁻¹): 3400, 1613, 1527

¹H NMR (DMSO-d₆, δ; 200 MHz): 6.42 (s, 1H); 71.12-7.29 (m, phenyl); 8.41 (9 s, 1H).

TGA thermogram: Weight loss at 100° C. (0.80%); 212° C. (5.4%); 227° C. (7.6%); 253° C. (16.7%); 258° C. (39.0%); and 336° C. (69.1%).

X-ray (powder) diffraction pattern:
(d, Å): 15.08, 11.18, 10.69, 7.92, 7.51, 7.14, 6.88, 6.66, 5.32, 5.00, 4.90, 4.41, 4.36, 4.30, 3.78, 3.72, 3.60, 3.50, 3.44; and
(2θ): 5.85, 7.89, 8.25, 11.14, 11.37, 12.37, 12.83, 13.26, 16.62, 17.72, 18.08, 20.11, 20.34, 20.59, 23.49, 23.86, 24.64, 25.41, 25.87).

### Example-2

### Preparation of Cefdinir (I)

### Step-1: p-Methoxybenzyl (Z)-(Tritylamino-2-thiazol-4yl)-2-Tritylxoyimino acetamido-3-vinyl-3-cephem-4-carboxylate

(Z)-(Tritylamino-2-thiazol-4yl)-2-Tritylxoyimino acetic acid (Compound II, 100 g, 0.144 moles) was suspended in dichloromethane and water was azeotropically distilled out until the moisture content of the mixture was below 0.06%. The solution was cooled to a temperature of between −20° C. to −25° C. and p-methoxybenzyl-7-amino-3-cephem-4-carboxylate p-toluenesulfonate salt (76.24 g, 0.147 moles) was added and the mixture was stirred at the same temperature for 10 minutes. Pyridine (19.25 ml, 0.272 moles) was added in one lot and the mixture was stirred for a further 10 minutes at a temperature of between −20° C. to −25° C. A solution of phosphorous oxychloride (22.19 ml, 0.238 moles) in dichloromethane (200 ml) was added, slowly over a period of 30 to 40 minutes while maintaining the temperature between −20° C. to −25° C. Thereafter, the mixture was agitated at that same temperature for 30 minutes. An aqueous solution of sodium hydroxide (50%, 77 ml) was added slowly over a period of 15 to 20 minutes. The temperature was allowed to rise to room temperature and the reaction mixture was concentrated to a low volume. The residual dichloromethane was completely stripped with toluene and the volume of toluene was concentrated to about 300 ml. The solution of p-Methoxybenzyl (Z)-(Tritylamino-2-thiazol-4yl)-2-Tritylxoyimino acetamido-3-vinyl-3-cephem-4-carboxylate in toluene was washed successively with water and saturated aqueous sodium chloride solution, and dried over anhydrous sodium sulfate.

### Step-2: Crude Cefdinir (I)

The solution of p-Methoxybenzyl (Z)-(Tritylamino-2-thiazol-4yl)-2-Tritylxoyimino acetamido-3-vinyl-3-cephem-4-carboxylate in toluene obtained in Step-1, was cooled to a temperature of between 5° C. to 10° C. and trifluoroacetic acid (350 ml) was added over a period of 30 to 40 minutes. After the addition was completed, the temperature was raised to 15° C. to 20° C. and the reaction mixture was stirred at that temperature for 3 hours. The temperature was further lowered to 0° C. to 5° C. and water (1050 ml) was slowly added over a period of 45 to 60 minutes while maintaining the temperature between 0° C. to 5° C. Thereafter, the mixture was agitated for 60 minutes and the solid that precipitated was filtered, washed with water, and dried under vacuum to give 95 g of crude cefdinir (I).

### Step-3: Purified Cefdinir (I)

A suspension of the crude cefdinir (95 g) obtained from Step-2 in water (1500 ml) was formed. This suspension was cooled to a temperature of between 5° C. to 10° C. and a solution of 10% aqueous ammonia was added until the pH reached between 6.3 to 6.5. The resulting solution was cooled to a temperature of between 4° C. to 6° C. and stirred for 15 minutes with activated carbon. The carbon was filtered off and the pH of the filtrate was adjusted to 2.3 to 2.5 by slowly adding 15% hydrochloric acid while maintaining a temperature of between 4° C. to 12° C. After the pH was adjusted, the mixture was stirred for 1 hour at a temperature of between 2° C. to 5° C. and the crystals were filtered, washed with water, and dried under vacuum to give 30 g (54%) of cefdinir (I). The cefdinir product had a purity of 99.7% and a water content of 5 to 7%.

The IR (KBr, cm⁻¹) spectrum included the following peaks: 3300, 1780, 1665, 1180, 1130. The following X-ray (powder) diffraction pattern was obtained.

| d-spacing (Å) | Angle (2θ) | Relative Intensity |
|---|---|---|
| 5.85 | 15.07 | 37.52 |
| 7.79 | 11.33 | 31.89 |
| 8.05 | 10.96 | 25.12 |
| 11.75 | 7.52 | 100.00 |
| 15.66 | 5.65 | 17.19 |
| 16.17 | 5.47 | 42.18 |
| 18.08 | 4.90 | 10.77 |
| 18.61 | 4.76 | 43.54 |
| 19.42 | 4.56 | 18.38 |
| 20.96 | 4.23 | 38.46 |
| 21.23 | 4.18 | 33.31 |
| 22.31 | 3.98 | 41.54 |
| 22.56 | 3.93 | 27.84 |
| 23.65 | 3.75 | 5.77 |
| 24.43 | 3.63 | 35.03 |
| 25.07 | 3.54 | 9.09 |
| 25.65 | 3.46 | 29.93 |
| 26.24 | 3.39 | 34.30 |
| 27.21 | 3.27 | 3.19 |
| 28.03 | 3.18 | 8.88 |
| 28.88 | 3.08 | 18.07 |
| 30.10 | 2.96 | 5.86 |
| 30.97 | 2.88 | 16.56 |
| 31.68 | 2.82 | 16.06 |
| 34.07 | 2.62 | 12.87 |
| 34.89 | 2.56 | 14.96 |
| 37.39 | 2.40 | 7.04 |
| 38.18 | 2.35 | 7.22 |
| 39.04 | 2.30 | 12.91 |
| 40.31 | 2.23 | 3.10 |
| 42.26 | 2.13 | 3.94 |
| 43.76 | 2.06 | 4.41 |
| 45.47 | 1.99 | 5.22 |
| 46.01 | 1.97 | 9.12 |
| 47.71 | 1.90 | 1.63 |
| 49.26 | 1.84 | 1.55 |
| 50.22 | 1.81 | 2.89 |
| 54.82 | 1.67 | 1.31 |
| 56.57 | 1.62 | 1.59 |
| 57.79 | 1.59 | 0.39 |
| 58.06 | 1.58 | 1.52 |

### Example-3

### Preparation of cefdinir (I)

### Step-1: p-Methoxybenzyl (Z)-(Tritylamino-2-thiazol-4yl)-2-Tritylxoyimino acetamido-3-vinyl-3-cephem-4-carboxylate

(Z)-(Tritylamino-2-thiazol-4yl)-2-Tritylxoyimino acetic acid (II, 100 g, 0.144 moles) was suspended in dichloromethane, water was azeotropically distilled out until the moisture content of the mixture was below 0.06%. The solution was cooled to a temperature of between −20° C. to −25° C. and p-methoxybenzyl-7-amino-3-cephem-4-carboxylate p-toluenesulfonate salt (76.24 g, 0.147 moles) was added. The mixture was stirred at the same temperature for 10 minutes. Pyridine (19.25 ml, 0.272 moles) was added in one lot and the mixture was stirred for an additional 10 minutes at a temperature of between −20° C. to −25° C. Slowly, over a period of 30 to 40 minutes, 2,4-diaminopyridine (38.92 g, 0.357 moles) and sodium bromide (37.12 g, 0.357 moles) were added, followed by a solution of phosphorous oxychloride (22.19 ml, 0.238 moles) in dichloromethane (200 ml), while maintaining the temperature between −20° C. to −25° C. Thereafter, the mixture was agitated at that same temperature for 30 minutes. An aqueous solution of sodium hydroxide (50%, 77 ml) was slowly added over a period of 15 to 20 minutes. The reaction mixture was allowed to rise to room temperature and concentrated down to a low volume. The residual dichloromethane was completely stripped with toluene and the volume of toluene was concentrated to about 300 ml. The solution of p-Methoxybenzyl (Z)-(Tritylamino-2-thiazol-4yl)-2-Tritylxoyimino acetamido-3-vinyl-3-cephem-4-carboxylate in toluene was washed successively with water and saturated aqueous sodium chloride solution, and dried over anhydrous sodium sulfate.

### Step-2: Crude Cefdinir

A solution of p-Methoxybenzyl (Z))-(Tritylamino-2-thiazol-4yl)-2-Tritylxoyimino acetamido-3-vinyl-3-cephem-4-carboxylate in toluene obtained in Step-1, was cooled to a temperature of between 5° C. to 10° C. and trifluoroacetic acid (350 ml) was added over a period of 30 to 40 minutes. After the addition was completed, the temperature was raised to 15° C. to 20° C. and the reaction mixture stirred at the same temperature for 3 hours. The temperature was lowered to 0° C. to 5° C. and water (1050 ml) was slowly added over a period of 45 to 60 minutes while maintaining the temperature between 0° C. to 5° C. Thereafter, the mixture was agitated for 60 minutes and the resulting precipitant was filtered, washed with water, and dried under vacuum to give 95 g of crude cefdinir (I).

### Step-3: Pure Cefdinir (I)

A suspension of the crude cefdinir (95 g) obtained from Step-2 in water (1500 ml) was prepared and cooled to a temperature of between 5° C. to 10° C. A solution of 10% aqueous ammonia was added until the pH reached between 6.3 to 6.5. The resulting solution was cooled to a temperature of between 4° C. to 6° C. and stirred for 15 minutes with activated carbon. The carbon was filtered off and the pH of the filtrate was adjusted to 2.3 to 2.5 through the slow addition of 15% hydrochloric acid, maintaining a temperature of between 4° C. to 12° C. After the pH was adjusted, the mixture was stirred for 1 hour at a temperature of between 2° C. to 5° C. and the crystals were filtered, washed with water, and dried under vacuum to give 28 g (50%) of cefdinir (I) having a purity of 99.7%, and a water content of 5 to 7%. The X-ray (powder) diffraction pattern was the same as that disclosed in Example 2. The IR spectrum (KBr, cm⁻¹) of the cefdinir product contained the following peaks: 3300, 1780, 1665, 1180, 1130.

### Example-4

### Preparation of Cefdinir (I)

### Step-1: (N, O)-bis-silyl-7-amino-3-vinyl-3-cephem-4-carboxylate

A solution containing 7-amino-3-vinyl-3-cephem-4-carboxylic acid (IX, 28 g, 0.123 moles) and dichloromethane (400 ml) was prepared. The mixture was cooled to 10° C. and bis silyl acetamide (BSA, 92 g, 0.453 moles) was added. The mixture was stirred at 25° C. for 60 minutes and thereafter cooled to −65° C. and kept at that temperature.

### Step-2: (Z)-(Tritylamino-2-thiazol-4yl)-2-Tritylxoyimino acetic acid chloride

(Z)-(Tritylamino-2-thiazol-4yl)-2-Tritylxoyimino acetic acid (II, 100 g, 0.144 moles) was suspended in dichloromethane (2800 ml) and water was azeotropically distilled out until the moisture content of the mixture was below 0.06%. Pyridine (19.25 ml, 0.272 moles) was added in one lot and the mixture was stirred for a further 10 minutes at a temperature of between −20° C. to −25° C. The solution was cooled to a temperature of between −20° C. to −25° C. A solution of phosphorous pentachloride (34 g, 0.163 moles) in dichloromethane (200 ml) was added and the solution kept at −20° C. to −25° C.

### Step-3: Protected Cefdinir

The solutions from Step-1 and 2 were mixed and 2,4-diaminopyridine (22.80 g, 0.245 moles) and sodium bromide (25.47 g, 0.245 moles) were added while maintaining the temperature between −20° C. to −25° C. The mixture was then agitated at the same temperature for 30 minutes. An aqueous solution of sodium hydroxide (50%, 77 ml) was slowly added over a period of 15 to 20 minutes. The temperature was allowed to rise to room temperature and the reaction mixture was concentrated down to a low volume. The residual dichloromethane was completely stripped with toluene and the resulting mixture was concentrated to about 300 ml. This solution of protected cefdinir in toluene was washed successively with water and saturated aqueous sodium chloride solution, and dried over anhydrous sodium sulfate.

### Step-4: Crude Cefdinir

The solution of protected cefdinir in toluene obtained in Step-3 was cooled to a temperature between 5° C. to 10° C. Trifluoroacetic acid (350 ml) was added over a period of 30 to 40 minutes. After the addition was completed, the temperature was raised to 15° C. to 20° C. and the reaction mixture was stirred at this same temperature for 3 hours. The temperature was further lowered to 0° C. to 5° C. and water (1050 ml) was slowly added over a period of 45 to 60 minutes while maintaining the temperature between 0° C. to 50° C. Thereafter, the mixture was agitated for 60 minutes and the resulting precipitant was filtered, washed with water, and dried under vacuum to yield 95 g of crude cefdinir (I).

### Step-4: Pure Cefdinir (I)

A suspension of the crude cefdinir (95 g) obtained from Step-3 in water (1500 ml) was formed and cooled to a temperature of between 5° C. to 10° C. A solution of 10% aqueous ammonia was added until the pH reached between 6.3 to 6.5. The resulting solution was cooled to a temperature of between 4° C. to 6° C. and stirred for 15 minutes with activated carbon. The carbon was filtered off and the pH of the filtrate was adjusted to 2.3 to 2.5 through the slow addition of 15% hydrochloric acid while maintaining a temperature of between 4° C. to 12° C. After the pH was adjusted, the mixture was stirred for 1 hour at a temperature between 2° C. to 5° C. and the crystals were filtered, washed with water, and dried under vacuum to give 33 g (54%) of cefdinir (I) having a purity of 99.7% and a water content of 5 to 7%. The X-ray (powder) diffraction pattern was the same as that disclosed in Example 2. The IR (KBr, cm⁻¹) spectrum included the following peaks: 3300, 1780, 1665, 1180, 1130.

### Example-5

### Preparation of Cefdinir (I)

### Step-1: (N,O)-bis-silyl-7-amino-3-vinyl-3-cephem-4-carboxylate

A solution of 7-amino-3-vinyl-3-cephem-4-carboxylic acid (IX, 28 g, 0.123 moles) in dichloromethane (400 ml) was cooled to 10° C. and bis silyl acetamide (BSA, 92 g, 0.453 moles) was added. The mixture was stirred at 25° C. for 60 minutes and thereafter cooled to −65° C. and kept at the same temperature.

### Step-2: (Z)-(Tritylamino-2-thiazol-4yl)-2-Tritylxoyimino acetic acid chloride

(Z)-(Tritylamino-2-thiazol-4yl)-2-Tritylxoyimino acetic acid (II, 100 g, 0.144 moles) was suspended in dichloromethane (2800 ml) and water was azeotropically distilled out until the moisture content of the mixture was below 0.06%. Pyridine (19.25 ml, 0.272 moles) was added in one lot and the mixture was stirred for a further 10 minutes at a temperature of between −20° C. to −25° C. A solution of phosphorous pentachloride (34 g, 0.163 moles) in dichloromethane (200 ml) was added and the solution was kept at −20° C. to −25° C.

### Step-3: Protected Cefdinir

The solutions from Step-I and Step-2 were mixed and agitated at a temperature between −20° C. to −25° C. for 30 minutes. An aqueous solution of sodium hydroxide (50%, 77 ml) was added slowly over a period of 15 to 20 minutes. The temperature was allowed to rise to room temperature and the reaction mixture was concentrated down to a low volume. The residual dichloromethane was completely stripped with toluene and the resulting solution was concentrated down to about 300 ml. The resulting solution of protected cefdinir in toluene was washed successively with water and saturated aqueous sodium chloride solution, and dried over anhydrous sodium sulfate.

### Step-4: Crude Cefdinir

The solution of protected cefdinir in toluene obtained in Step-3, was cooled to a temperature of between 5° C. to 10° C. and trifluoroacetic acid (350 ml) was added over a period of 30 to 40 minutes. After the addition was completed, the temperature was raised to 15° C. to 20° C. and the reaction mixture was stirred at the same temperature for 3 hours. The temperature was further lowered to 0° C. to 5° C. and water (1050 ml) was slowly added over a period of 45 to 60 minutes maintaining the temperature between 0° C. to 5° C. Thereafter, the mixture was agitated for 60 minutes and the resulting precipitant was filtered, washed with water, and dried under vacuum to give 95 g of crude cefdinir (I).

### Step-5: Pure cefdinir (I)

A suspension of the crude cefdinir (95 g) obtained from Step-3 in water (1500 ml) was formed, cooled to a temperature of between 5° C. to 10° C. and a solution of 10% aqueous ammonia was added until the pH reached between 6.3 to 6.5. The resulting solution was cooled to a temperature of between 4° C. to 6° C. and stirred for 15 minutes with activated carbon. The carbon was filtered off and the pH of the filtrate was adjusted to 2.3 to 2.5 through the slow addition of 15% hydrochloric acid, maintaining a temperature of between 4° C. to 120° C. After the pH was adjusted, the mixture was stirred for 1 hour at a temperature of between 2° C. to 5° C. and the crystals were filtered, washed with water, and dried under vacuum to give 32 g (52.4%) of cefdinir (I) having a purity of 99.7%, a water content of 5 to 7%, and yielding the X-ray (powder) diffraction pattern provided in Example 2. The IR (KBr, cm⁻¹) spectrum of the cefdinir product was: 3300, 1780, 1665, 1180, 1130.

1. A stable, bioavailable crystalline form of cefdinir of formula (I) 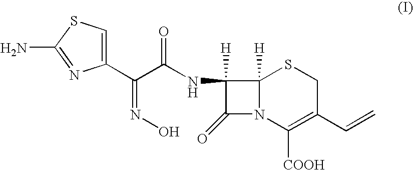
characterized by a water content of between about 6.0 to about 7.0%; and having an X-ray (powder) diffraction pattern comprising the following d-spacing values within about 5%:

| d-spacing (Å) | Relative Intensity |
|---|---|
| 15.07 | 37.52 |
| 11.33 | 31.89 |
| 10.96 | 25.12 |
| 7.52 | 100.00 |
| 5.65 | 17.19 |
| 5.47 | 42.18 |
| 4.90 | 10.77 |
| 4.76 | 43.54 |
| 4.56 | 18.38 |
| 4.23 | 38.46 |
| 4.18 | 33.31 |
| 3.98 | 41.54 |
| 3.75 | 5.77 |
| 3.63 | 35.03 |
| 3.54 | 9.09 |
| 3.46 | 29.93 |
| 3.39 | 34.30 |
| 3.27 | 3.19 |
| 3.18 | 8.88 |
| 3.08 | 18.07 |
| 2.96 | 5.86 |
| 2.88 | 16.56 |
| 2.82 | 16.06 |
| 2.62 | 12.87 |
| 2.56 | 14.96 |
| 2.40 | 7.04 |
| 2.30 | 12.91 |
| 1.99 | 5.22 |
| 1.97 | 9.12 |

2. A process for the preparation of a crystalline form of cefdinir comprising:
(a) reacting a crystalline compound of formula (II) 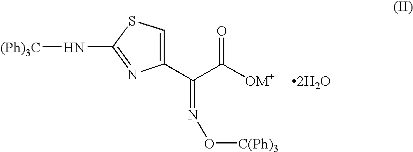
wherein M is an alkali metal, with a chlorinating or brominating agent in the presence of a water-immiscible organic solvent and in the presence of an organic base to produce a compound of formula (IV), 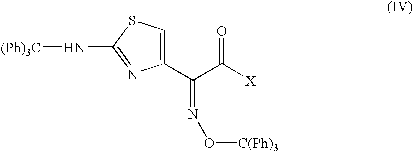
wherein X is Cl or Br,
(b) reacting the compound of formula (IV) with a compound of formula (III), 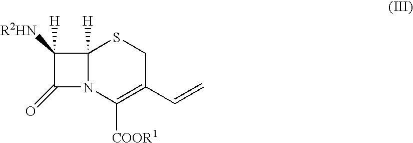
wherein R¹ is a trialkylsilyl protective group or a carboxylic acid protective group; and R² is a trialkyl silyl group or a organic sulfonic acid, to produce a compound of formula (VII), 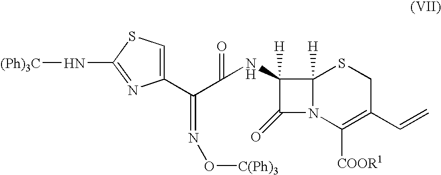
wherein R¹ is a trialkylsilyl protective group or a carboxylic acid protective group,
(c) placing the compound of formula (VII) in a hydrocarbon solvent,
(d) reacting the compound of formula (VII) with a second acid to remove the protective groups and produce a crude cefdinir product (I), either as a free base or as a salt of said second acid, and
(e) crystallizing the crude cefdinir product (I) to produce a crystalline form of cefdinir (I).

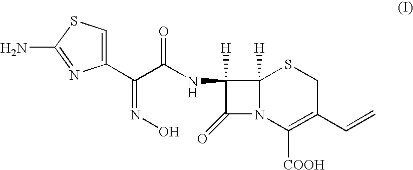

3. A process according to claim 2 wherein said crystallization comprises the steps of:
(i) dissolving the crude cefdinir product (I) in water at a pH between about 6.3 to about 7.0;
(ii) modifying the pH of the reaction mixture to between about 2.3 to about 2.5 at a temperature of between about 0° C. to about 12° C. using an acid to effect crystallization;
(iii) agitating the crystals at a temperature of between about 0° C. to about 12° C. for a period of between about 30 minutes to about 120 minutes; and
(iv) filtering the crystals and drying

4. A process according to claim 2 wherein the crystallization further comprises the step of decolorizing the solution by treatment with activated carbon and filtering off the carbon.

5. A process according to claim 2, wherein the chlorinating agent is used and said chlorinating agent comprises a compound selected from the group consisting of thionyl chloride, sulfury chloride, phosphorous trichloride, phosphorous pentachloride, phosphorous oxychloride, or oxalyl chloride.

6. A process according to claim 2, wherein the brominating agent is used and said brominating agent comprises a compound selected from the group consisting of thionyl bromide, phosphorous tribromide, or phosphorous pentabromide.

7. A process according to claim 2 wherein the chlorinating agent is used in molar proportions of about 1.0 to about 3.0 moles per mole of the compound of formula (II).

8. A process according to claim 2 wherein the brominating agent is used in molar proportions of about 1.0 to about 3.0 moles per mole of the compound of formula (II).

9. A process according to claim 2, wherein the water-immiscible organic solvent comprises a compound selected from the group consisting of a chlorinated hydrocarbon or an aromatic hydrocarbon.

10. A process according to claim 9 wherein the water-immiscible organic solvent comprises a chlorinated hydrocarbon comprising at least one of dichloromethane or dichloroethane.

11. A process according to claim 9 wherein the water-immiscible organic solvent comprises an aromatic hydrocarbon comprising at least one of benzene, toluene, or xylene.

12. A process according to claim 2, wherein the organic base comprises a compound selected from the group consisting of dimethylamine, diethylamine, trimethylamine, triethylamine, triisopropylamine, tertiarybutylamine, dimethylaniline, diethylaniline, pyridine, dicyclohexylamine, DBN, DBU, and N-methylmorpholine, or mixtures thereof.

13. A process according to claim 2, wherein the organic base is used in molar proportions of about 1.0 to about 3.0 moles per mole of the compound of formula (II).

14. A process according to claim 2, wherein the reaction of the compound of formula (II) with the compound of formula (III) is conducted at a temperature of about −1° C. to about −650 C.

15. A process according to claim 2, wherein the reaction of the compound of formula (II) with the compound of formula (III) is conducted at a temperature of about −25° C. to about −35° C.

16. A process according to claim 2, wherein R¹ in the compound of formula (III) comprises p-methoxybenzyl or benzhydryl.

17. A process according to claim 2, wherein R² of the compound of formula formula (III) comprises p-toluenesulfonic acid or methanesulfonic acid.

18. A process according to claim 2, wherein the hydrocarbon solvent used in step (c) comprises a compound selected from the group consisting of benzene, toluene, xylene, or mixtures thereof.

19. A process according to claim 2, wherein the second acid comprises a compound selected from the group consisting of trifluoroacetic acid, methanesulfonic acid, hydrochloric acid, formic acid, or mixtures thereof.

20. A process for the preparation of a crystalline form of cefdinir comprising:
(a) reacting a crystalline compound of formula (II) 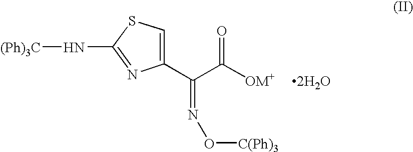
wherein M is an alkali metal, with a chlorinating agent in the presence of a dialkylaminopyridine and an alkali metal bromide in the presence of a water-immiscible organic solvent and in the presence of an organic base to form the corresponding acid bromide of formula (IV), wherein X is Br, 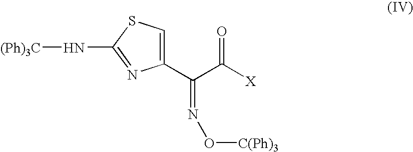
(b) reacting the compound of formula (IV) with a compound of formula (III), 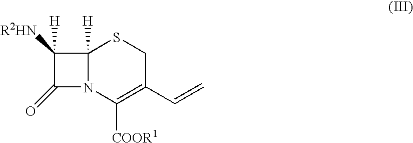
wherein R¹ is a trialkylsilyl protective group or a carboxylic acid protective group; and R² is a trialkyl silyl group or a organic sulfonic acid, to produce a compound of formula (VII), 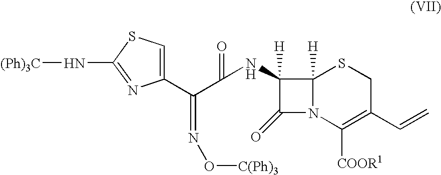
wherein R¹ is a trialkylsilyl protective group or a carboxylic acid protective group,
(c) placing the compound of formula (VII) in a hydrocarbon solvent,
(d) reacting the compound of formula (VII) with a second acid to remove the protective groups and produce a crude cefdinir product (I), either as a free base or as a salt of said second acid, and
(e) crystallizing the crude cefdinir product (I) to produce a crystalline form of cefdinir (I).

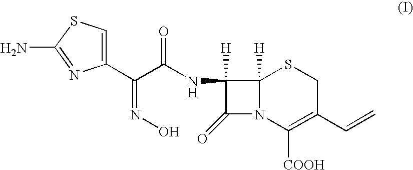

21. A process according to claim 20 wherein said crystallization comprises the steps of:
(i) dissolving the crude cefdinir product (I) in water at a pH between about 6.3 to about 7.0;
(ii) modifying the pH of the reaction mixture to between about 2.3 to about 2.5 at a temperature of between about 0° C. to about 12° C. using an acid to effect crystallization;
(iii) agitating the crystals at a temperature of between about 0° C. to about 12° C. for a period of between about 30 minutes to about 120 minutes; and
(iv) filtering and drying the crystals.

22. A process according to claim 20 wherein the dialkylaminopyridine comprises a compound selected from the group consisting of dimethylaminopyridine or diethylaminopyridine, or mixtures thereof.

23. A process according to claim 20, wherein the dialkylaminopyridine is used in molar proportions of about 1.0 to about 3.0 moles per mole of the compound of formula (II).

24. A process according to claim 20, wherein the alkali metal bromide comprises a compound selected from the group consisting of sodium bromide, potassium bromide, lithium bromide, or mixtures thereof.

25. A process according to claim 20, wherein the alkali metal bromide is used in molar proportions of about 1.0 to about 3.0 moles per mole of the compound of formula (II).

26. A stable and bioavailable antimicrobial pharmaceutical composition comprising an antimicrobially effective amount of the stable, bioavailable crystalline cefdinir of claim 1.

27. A pharmaceutical composition according to claim 26 wherein the cefdinir is in an admixture with a pharmaceutically acceptable carrier.

28. A pharmaceutical composition according to claim 27, wherein the pharmaceutically acceptable carrier comprises a compound selected from the group consisting of disintegrants, lubricants, suspending agents, thickening agents, preservatives, bulking agents, flavoring agents, pH modifiers, or mixtures thereof.

29. A pharmaceutical composition according to claim 26, which is in the form of a capsule, tablet, or suspension.

30. A pharmaceutical composition according to claim 26, which is for oral administration.

31. A pharmaceutical composition according to claim 26, which exhibits the stability and bioequivalence of the Omicef® formulation of cefdinir.
